(19) United States
(12) Patent Application Publication
(10) Pub. No.: US 2022/0270712 A1
(43) Pub. Date: Aug. 25, 2022

(54) SYSTEMS AND METHODS FOR AUTOMATED ANALYSES OF A BIOLOGICAL SAMPLE

(71) Applicants: Rutgers, The State University of New Jersey, New Brunswick, NJ (US); National University of Ireland Maynooth, Maynooth University, Maynooth (IE)

(72) Inventors: Catherine M. Grgicak, New Brunswick, NJ (US); Desmond S. Lun, New Brunswick, NJ (US); Kenneth R. Duffy, Dublin (IE)

(21) Appl. No.: 17/669,790

(22) Filed: Feb. 11, 2022

Related U.S. Application Data
(60) Provisional application No. 63/149,498, filed on Feb. 15, 2021.

(51) Int. Cl. G16B 40/30; G16B 30/00; G16B 5/20; G16B 20/20

(52) U.S. Cl. CPC G16B 40/30; G16B 20/20; G16B 5/20; G16B 30/00

(57) ABSTRACT

Systems and methods of the present disclosure enable automated analyses of a biological sample using a processing system by receiving signal profiles of each allele of a set of cells in the sample. A set of allele vectors are determined based on a mapping of the magnitude of the measurement of each signal profile at each locus to an index location. A set of cell vectors is generated by concatenating each allele vector of each cell. A cluster model is utilized to generate clusters of the signal profiles based on the set of cell vectors to represent contributors. A first likelihood of a target contributor matching a contributor and a second likelihood of the target contributor not matching any contributor are determined by comparing the target signal profile to each cluster. A likelihood ratio is determined from a ratio of the first likelihood and the second likelihood.

### CROSS-REFERENCE TO RELATED APPLICATION

This application claims the benefit of and priority to U.S. Provisional Application No. 63/149,498, filed Feb. 15, 2021, which is incorporated herein by reference in its entirety.

### STATEMENT OF RIGHTS TO INVENTIONS MADE UNDER FEDERALLY SPONSORED RESEARCH

This invention was made with government support under Grant No. NIJ2018-DU-BX-0185 awarded by the National Institute of Justice. The government has certain rights in the invention.

### FIELD OF INVENTION

The present disclosure generally relates to detection, isolation, and/or analysis of biological molecules of interest. The disclosure provides embodiments with applications in, for example, the fields of genetics, bioinformatics, molecular biology, high-throughput screening, diagnostics, statistics, and the like.

### BACKGROUND

It is therefore an object of this disclosure to improve on forensic DNA mixture interpretation in the forensic domain, assessing number of species in a mixture in the environmental chemistry/biology domain, bone-marrow transplant assessments. For example, some forensic DNA technologies are prone to inconsistent results in the presence of multiple contributors.

For example, some methods may be used to infer the number of contributors and weight of evidence from a group of single cells using qualitative data, i.e., the number of times a peak exceeds a signal threshold across a plurality of cells, but do not use quantitative data, i.e., the peak heights obtained. These methods are not suitable for single-cell samples since they exhibit high levels of allele non-detection and high expressions of artifacts such as stutter—a frequently occurring artifact that often results in one additional peak one repeat unit less or greater than the allele.

### BRIEF DESCRIPTION OF FIGURES

FIG. 1 depicts a proportion of samples originating from the known number of contributors versus the number of peaks ≥1 RFU at a locus for all mixture samples in a set of mixture samples. In no instance are greater than eight detections at allele positions observed at a locus, despite the presence of five-person genotype combinations in the database according to aspects of embodiments of the present disclosure.

FIG. 2 illustrates three representative loci from three cells sampled from a 2-person admixture of epithelial cells from an unknown, or evidentiary type, sample according to aspects of embodiments of the present disclosure.

FIG. 3 illustrates: Top panel: The green channel of a single cell DNA profile from picopetting coupled with a forenicGem lysis and Identifiler Plus amplification according to aspects of embodiments of the present disclosure. Bottom panel: The profile obtained when a portion of the sample is pipetted though no cell is captured in the tip.

FIG. 4. Peak height (RFU) distributions of STR peaks obtained for the four extraction kits according to aspects of embodiments of the present disclosure.

FIG. 5 illustrates Histograms of the ‘Number of recovered heterozygous alleles’ from 136 single cell samples for Persons 01, 05 and 06 according to aspects of embodiments of the present disclosure. Maximum number of recoverable alleles, 34, per EPG. Histogram of number of alleles above an RFU of 30 per EPG fractionated by person tested. Best-fit distribution of the number of recovered alleles if allele dropout was independent of the cell and locus. These data indicate that during inference the dropout cannot be modeled as a cell independent random variable with fixed probability for these sample types.

FIG. 6 illustrates Stutter Ratio (SR) versus the peak height of the True allele in RFU (log-scale) for 34 single cells using four distinct extraction kits (f=ForensicGem; p=PicoPure; s=LysePrep;v=DirectPCR) for Person 01 according to aspects of embodiments of the present disclosure. The vertical range has been clipped at a SR of 5, resulting in 5 larger SRs not being shown.

FIG. 7A illustrates a block diagram of an illustrative method for clustered single cell DNA forensics according to embodiments of the present disclosure.

FIG. 7B illustrates a block diagram of an illustrative system for clustered single cell DNA forensics according to embodiments of the present disclosure.

FIG. 8 illustrates a block diagram of an illustrative system for clustering single cell signal profiles for clustered single cell DNA forensics according to embodiments of the present disclosure.

FIG. 9 illustrates a block diagram of another illustrative system for clustering single cell signal profiles for clustered single cell DNA forensics according to embodiments of the present disclosure.

FIG. 10 illustrates a block diagram of an illustrative system for testing DNA sequence hypotheses against clustered single cell signal profiles for clustered single cell DNA forensics according to embodiments of the present disclosure.

FIG. 11 illustrates a block diagram of an illustrative visualization engine for visualizing clustered single cell DNA forensics according to embodiments of the present disclosure.

FIG. 12 illustrates allele fluorescent measurements from electropherogram (EPG) of a single-cell according to aspects of embodiments of the present disclosure.

FIG. 13 illustrates the mapping and conversion of allele fluorescent measurements into a concatenated vector, e.g., using a loci-index map as described above according to aspects of embodiments of the present disclosure.

FIG. 14 illustrates an example distribution of similarity or dissimilarity according to cosine distances between vectors of signal profiles where the dotted lines indicate self-self dissimilarity and the solid lines indicate self-non-self dissimilarity according to aspects of embodiments of the present disclosure.

FIG. 15A depicts example illustration of a correct clustering result according to aspects of embodiments of the present disclosure.

FIG. 15B depicts example illustration of an overclustering result according to aspects of embodiments of the present disclosure.

FIG. 15C depicts example illustration of a misclustering result according to aspects of embodiments of the present disclosure.

FIG. 16 depicts an example illustration of admixtures having multiple clustered contributors according to aspects of embodiments of the present disclosure.

FIG. 17 illustrates an overview of allele signals for a (2;2;2;2;32) simulated admixture according to aspects of embodiments of the present disclosure.

FIG. 18 illustrates an Mclust cluster 5 according to aspects of embodiments of the present disclosure.

FIG. 19 illustrates an Mclust cluster 1 according to aspects of embodiments of the present disclosure.

FIG. 20 depicts a block diagram of an exemplary computer-based system and platform 2000 in accordance with one or more embodiments of the present disclosure.

FIG. 21 depicts a block diagram of another exemplary computer-based system and platform 2100 in accordance with one or more embodiments of the present disclosure.

FIG. 22 illustrates schematics of an exemplary implementations of the cloud computing/architecture.

FIG. 23 illustrates schematics of another exemplary implementations of the cloud computing/architecture.

FIG. 24 illustrates an exemplary single-cell signal profile using capillary electrophoresis (CE) to produce an electropherogram (EPG).

FIG. 25 provides an exemplary single-cell signal profile using NextGen Sequencing (NGS) to produce a readout.

FIG. 26 depicts an example distribution of Cosine Distances of EPGs from the same genotype (Self-Self) and of EPGs from one genotype to another (Self-Non-Self) according to aspects of embodiments of the present disclosure.

FIG. 27 depicts, for Persons 01, 05 and 06, an example dendrogram that results from agglomerative clustering according to aspects of embodiments of the present disclosure, where the vertical distances relate to the dissimilarity between all objects beneath that branch and the other objects connected by that branch. Blue, Green and Red branches correctly represent Person 05, 01 and 06, respectively. The black clusters represent low-quality DNA EPGs, which are dissimilar from the other EPGs.

FIG. 28A depicts an example distribution of Cosine Distances of EPGs from the same genotype (Self-Self) and of EPGs from one genotype to another (Self-Non-Self) for EPGs with a total RFU>15,000 according to aspects of embodiments of the present disclosure.

FIG. 28B depicts an example dendrogram that results from agglomerative clustering on all data according to aspects of embodiments of the present disclosure where the vertical distances relate to the distance between all objects beneath that branch and the other objects connected by that branch.

FIG. 29 depicts an example clustering of a 5-cell, low-copy cellular admixture through subjected to the single-cell pipeline according to aspects of embodiments of the present disclosure.

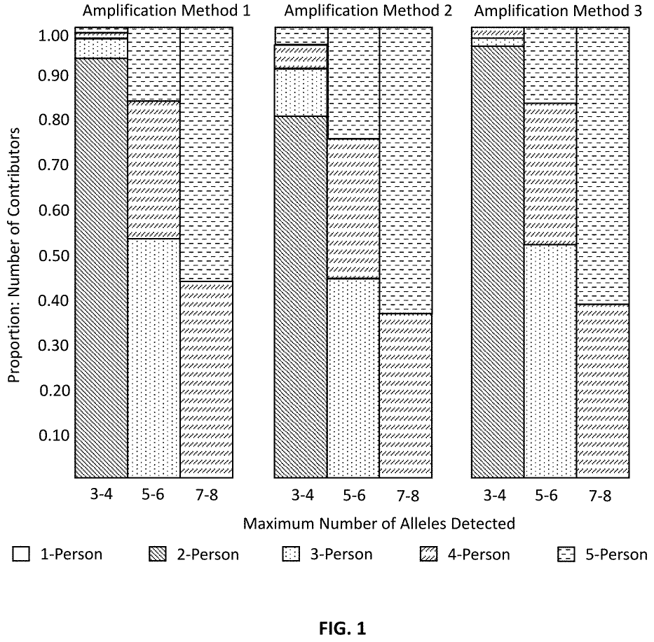

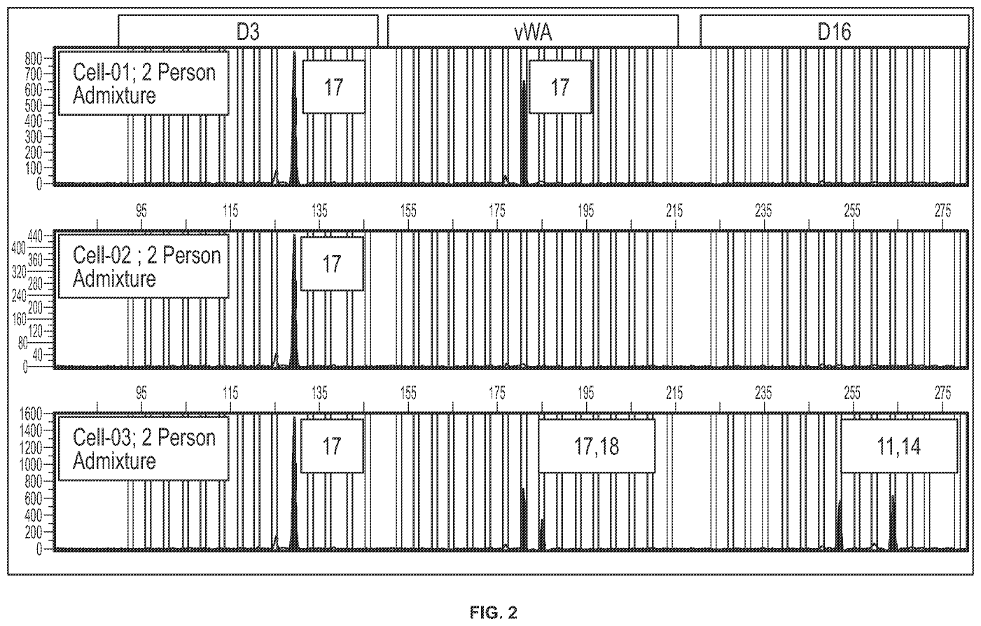

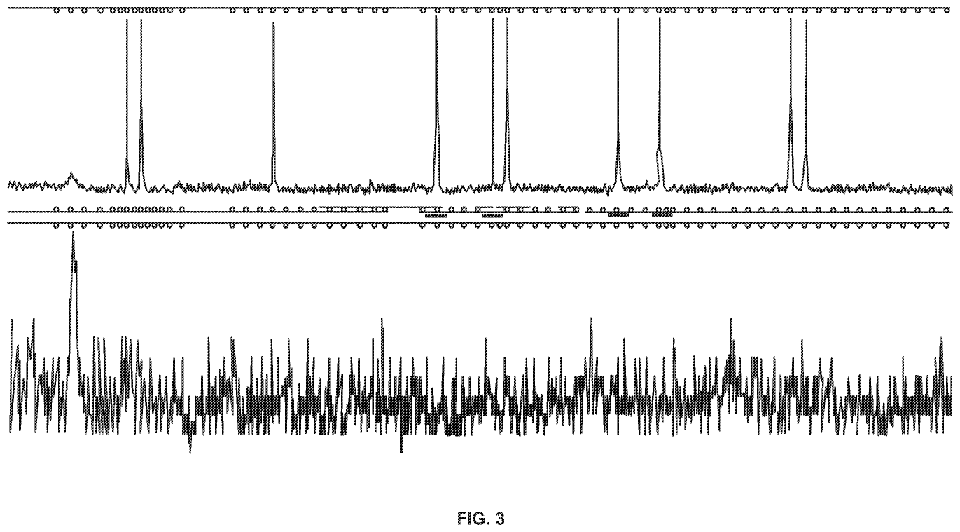

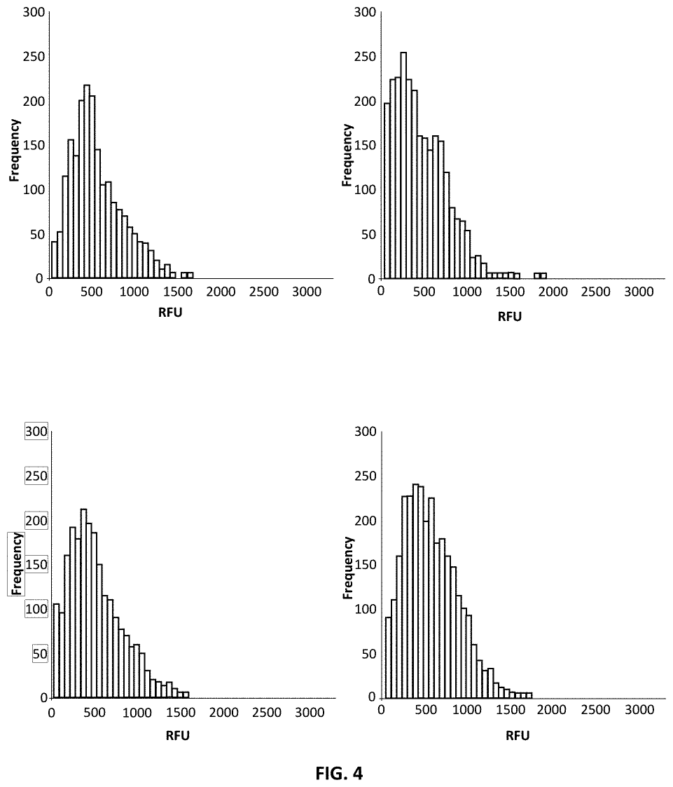

### DETAILED DESCRIPTION

Detailed embodiments of the present disclosure are disclosed herein; however, it is to be understood that the disclosed embodiments are merely illustrative of the disclosure that may be embodied in various forms. In addition, each of the examples given in connection with the various embodiments of the disclosure is intended to be illustrative, and not restrictive.

All terms used herein are intended to have their ordinary meaning in the art unless otherwise provided. All concentrations are in terms of percentage by weight of the specified component relative to the entire weight of the topical composition, unless otherwise defined.

As used herein, “a” or “an” shall mean one or more. As used herein when used in conjunction with the word “comprising,” the words “a” or “an” mean one or more than one. As used herein “another” means at least a second or more.

As used herein, all ranges of numeric values include the endpoints and all possible values disclosed between the disclosed values. The exact values of all half integral numeric values are also contemplated as specifically disclosed and as limits for all subsets of the disclosed range. For example, a range of from 0.1% to 3% specifically discloses a percentage of 0.1%, 1%, 1.5%, 2.0%, 2.5%, and 3%. Additionally, a range of 0.1 to 3% includes subsets of the original range including from 0.5% to 2.5%, from 1% to 3%, from 0.1% to 2.5%, etc. It will be understood that the sum of all weight % of individual components will not exceed 100%.

By “consist essentially” it is meant that the ingredients include only the listed components along with the normal impurities present in commercial materials and with any other additives present at levels which do not affect the operation of the embodiments disclosed herein, for instance at levels less than 5% by weight or less than 1% or even 0.5% by weight.

In some embodiments, the methods and systems of the disclosure may be applied to forensic samples that typically contain biological material (e.g., cells) of an unknown number of unknown individuals or contributors. Analyzing individual cells also provides additional data as to the cell type in addition to the contributor. Some embodiments of the disclosure provide for methods of analyzing forensic DNA having the steps of: 1) collecting samples containing cells; 2) separating different cell types; 3) extracting nucleic acids (e.g., DNA, RNA) from each cell; 4) amplifying biomolecular markers or genetic markers, such as short tandem repeats (STRs), of the extracted nucleic acids; 5) separating the biomolecular markers (e.g., STR amplicons) using separation techniques (e.g., capillary electrophoresis) that produce a signal; 6) detecting the signals comprising signal intensity, sizing, and allele assignment; and 7) interpreting the signals.

### Sample Preparation and Detection

Embodiments of the disclosure directed to DNA analysis may begin with obtaining and preparing samples for use in methods of amplifying biomolecular markers in the nucleic acid sequences of the sample, and in some embodiments, amplification of DNA or the entire genome of a single cell, chromosomes, or fragments thereof. DNA typing, DNA profiling, or genotyping are methods of isolating and identifying sequences of variable DNA or biomolecular markers that are repeated within the base-pair sequence of DNA in genes. Since each individual has a unique pattern of these highly variable DNA sequences, the likelihood of a sample belonging to a particular individual may be determined.

In forensics, a sample may have cells from, for example, skin, hair, blood, or body fluids (e.g., saliva, urine, semen). Oftentimes samples may be found on fabrics or textiles or surfaces (e.g., guns, knives, glassware, utensils, flooring) and should be properly collected and stored until analysis may occur. Traditional methods of forensic analyses of bulk mixtures produce one genetic profile from several cells and/or cell types. However, the bulk mixture interpretation and computation of match-statistic when the number of contributors in a sample is, for example, greater than 4 (e.g., 5, 6, 7, 8, 9, 10, 15) is computationally intensive because there are too many genotype combinations and/or includes degraded, damaged or inhibited DNA. DNA degradation or PCR inhibition originate from numerous underlying mechanisms, the characteristic is one of decreasing signal intensity as the molecular weight of the DNA fragment increases (i.e., referred to as ‘sloping-effect’). In addition, as the number of contributors in a sample increases the likelihood that a random person may have contributed to the DNA increases, resulting in a decrease in the weight-of-evidence (“WOE”) for actual contributors. Thus, in addition to samples containing contributors greater than 4 being computationally burdensome, the signal generated from these types of admixtures would be so convoluted that the data are less informative Moreover, as the traditional technique produces combined information on all cells in a sample, the information cannot be post-processed for determination of a match-statistic per cell-type. In contrast, one of the embodiments of the disclosure may be directed to single-cell analysis which allows for the computation of match-statistic for samples containing any number of contributors, including for example, more than 4 contributors since genotype combinations need not be considered in this analysis. Regardless of the number of contributors, profiles may be determined for individual cell types. See, e.g., Findlay et al. Nature, 389:555-556, 1997. Therefore, single-cell analysis allows for determining the likelihood of observing the data from different cell types given specified individuals supplied the DNA. For example, an analysis of whether a potential suspect contributed to blood cells versus epithelial or skin cells may be determined.

In single-cell analysis embodiments, individual cells first need to be isolated and/or identified. The single-cell methods of the disclosure occur by separating each cell prior to the extraction step. Non-limiting cell isolation techniques include density gradient centrifugation, membrane filtration, and microchip-based capture techniques that rely on physical properties such as but not limited to size, density, electric changes, and the like. Other cell isolation or separation techniques may be based on cellular biological characteristics, including but not limited to, affinity methods (e.g., affinity solid matrix using beads, plates, fibers, and the like) fluorescence-activated cell sorting (FACS), and magnetic-activated cell sorting (MACS). For example, Becton, Dickinson and Company cell sorting systems (e.g., BD FACSAria III™ Cell Sorter) may isolate single cells separating different cell types from thousands of cells in a population using various surface markers based on fluorescence and collecting charged cells of interest. Other types of high throughput cell isolation or separation methods may include MACS and microfluidic techniques. In one embodiment magnetic beads conjugated with one half of a protein binding pair, such as but not limited to, antibodies, streptavidins, enzymes, lectins, where the other half of the binding pair may be specific proteins on different cells of interest. Cell type isolation may occur when a mixed population of cells is subjected to an external magnetic field and charge separation. Another embodiment utilizes microfluidics to sort different cell types of interest. Different cell sorting microfluidic techniques may be based on, but not limited to, cell-affinity chromatography, physical characteristics of cells, immunomagnetic beads, and dielectric differences of different cell types.

Briefly, nucleic acid extraction involves a procedure that isolates nucleic acids from the nucleus of cells (see, e.g., Roberts, K. et al. “Molecular Cloning A Laboratory Manual Fourth Edition.” (2015)). Cells from a sample may release nucleic acids (e.g., DNA, RNA) by first breaking the cells open or lysing the cell membrane. Lysis buffer may comprise a detergent and a salt solution. A detergent may be added to break down lipids found in the cell membrane and nuclei, thereby releasing nucleic acids. The nucleic acids may be separated from proteins and other cellular debris by using protein enzymes such as proteases and/or filtrating the sample and precipitated by adding an alcohol since nucleic acids are insoluble in salt and alcohol. The nucleic acids may be further purified by resuspension in an alkaline buffer. DNA analysis, as well as RNA converted to cDNA by reverse transcription, may be performed after extraction. Non-limiting commercially available kits and known extraction techniques include: QIAamp® DNA Investigator Kit (Qiagen), DNA IQ™ System Kit (Promega), AutoMate Express™ Forensic DNA Extraction System (Applied Biosystems), Chelex 100 chelating resin).

Since extracted nucleic acid samples may be limited in quantity or size producing only small amounts of DNA (e.g., as little as 0.03 ng), damaged, or degraded, amplifying the DNA allows for sufficient amounts of DNA to be produced for further analysis. DNA analysis methods for distinguishing the genotype of an individual or subject to at least one or more individuals is referred to as genotyping, which identifies the biomolecular markers (e.g., alleles) of an individual. Non-limiting examples of amplifying and genotyping methods include: polymerase chain reaction (PCR), DNA sequence analysis (e.g., high-throughput sequencing, Next Gen sequencing (NGS), massive parallel signature sequencing (MPSS), multiplex sequencing), restriction fragment length polymorphism (RFLP) analysis, random amplified polymorphic detection (RAPD), amplified fragment length polymorphism detection (AFLPD), allele specific oligonucleotide (ASO) probes, hybridization to DNA microarrays or beads, and the like. Amplification methods such as those based on PCR may be used to amplify non-coding regions of DNA having a sequence of 2-400 base pairs that are repeated numerous times. These biomolecular markers for individual identification, may be, for example, sequences of DNA, such as those having a length of 2 base pairs (bp) to 400 base pairs, including single nucleotide polymorphisms (SNPs) and short tandem repeats (STRs) (e.g., 2 bp-14 bp, 2 bp-12 bp, 2 bp-10 bp, 2 bp-8 bp, 2 bp-6 bp, 2 bp-4 bp). Next Generation Sequencing (NGS) allows for SNP detection, which may lead to SNP genotyping. SNPs often occur within and outside of an STR repeat, so sub-divisions of an STR like may be produced (e.g., alleles 15a and 15b, where allele 15a is an STR of 15 repeats and an A/G/C/T in position x, while allele 15b of an STR is still 15 repeats but with another nucleotide in position x). SNP markers may be used to further parse out STR information or use SNPs on their own. The number of such sequences or units that are repeated varies among individuals allowing for the identification and potential likelihood that the biological markers are associated with a particular individual. The biological markers or nucleic acid sequences (e.g., SNPs, STRs) may be repeatedly amplified to produce thousands of copies of the STRs. Non-limiting examples of biological markers or loci may include, CSF1PO, D10S1248, D12ATA63, D12S391, D13S317, D16S539, D18S51, D19S433, D1S1656, D21S11, D22S1045, D2S1338, D2S441, D3S1358, D5S818, D7S820, D8S1179, FGA, TH01, TPOX, VWA, SE33, amelogenin (AMEL) gene which identifies an individual's sex; Y-chromosome STR markers: DYS385 (including DYS385a, DYS385b), DYS388, DYS389 (including, e.g., DYS389i, DYS389ii), DYS390, DYS391, DYS392, DYS393 (aka DYS395), DYS394 (aka DYS19), DYS413, DYS425, DYS426, DYS434, DYS435, DYS436, DYS437, DYS438, DYS439 (aka Y-GATA-A4), DYS441, DYS442, DYS443, DYS444, DYS445, DYS446, DYS447, DYS448, DYS449, DYS450, DYS452, DYS453, DYS454, DYS455, DYS456, DYS458, DYS459 (including e.g., DYS459a, DYS459b), DYS460 (aka Y-GATA-A7.1), DYS461 (aka Y-GATA-A7.2), DYS462, DYS463, DYS464 (including, e.g., DYS464a, DYS464b, DYS464c, DYS464d, DYS464e, DYS464f), DYS481, DYS485, DYS487, DYS490, DYS494, DYS495, DYS497, DYS504, DYS505, DYS508, DYS518, DYS520, DYS522, DYS525, DYS531, DYS532, DYS533, DYS534, DYS540, DYS549, DYS556, DYS557, DYS565, DYS570, DYS572, DYS53, DYS575, DYS576, DYS578, DYS589, DYS590, DYS594, DYS607, DYS612, DYS614, DYS626, DYS627, DYS632, DYS635 (aka Y-GATA-C4), DYS636, DYS638, DYS641, DYS643, DYS710, DYS714, DYS716v717, DYS724, DYS725, DYS726, DYF371, DYF385S1, DYF387S1a/b, DYF397, DYF399, DYF401, DYF406S1, DYF408, DYF411, DXYS156, YCAII (including, e.g., YCAIIa, YCAIIb), Y-GATA-H4, Y-GATA-A10, Y-GGAAT-1B07, etc.; X-chromosome STR markers: DXS10011, DXS10066 (aka Penta X-16), DXS10067 (aka Penta X-12), DXS10068 (aka Penta X-13), DXS10069 (aka Penta X-15), DXS10074, DXS 10075, DXS10079, DXS10129 (Penta X-10), DXS10130 (aka Penta X-3), DXS10131, DXS10132 (aka Penta X-17), DXS10133 (Penta X-18), DXS807, DXS7132, DXS7423, DXS8377, DXS981, HPRTB. However, any nucleic acid sequence that uniquely identify individuals may be used as a marker.

In some embodiments, the nucleic acid sequence markers are not limited to STR loci, but may include, for example, SNPs, combinations of SNPs, STRs, or combinations of STRs and SNPs. Moreover, the method may vary as long as the signal intensity information for a given allele may be attained, where the form of the allele may be length/sequence. In some embodiments, STR length or allele information may be supplemented by additional SNP information which can be used in the clustering or likelihood calculations as well as combinations of SNPs within a given DNA fragment. The technology is, therefore, not limited to length variation and may include sequence variation or a combination thereof.

Some embodiments of the disclosure may produce and provide signal profiles showing signal intensity as a function of fragment length of each amplified DNA fragment, thereby indicating how many copies of a particular biomolecular marker the fragment contains. The analysis of a sample may result in any number “n” of signal profiles comprising a signal intensity compared to genetic information (e.g., nucleic acid fragment length) for each cell in the sample. The types of signals may vary depending on the methodology used. For example, the signal may be produced by fluorescence, chemiluminescence, current or potential, radioactivity, detectable dyes (e.g., ethidium bromide). In the single-cell analysis method embodiment, the signal may be generated from an individual cell and produce multiple signal profiles, one for each cell. Whereas in the traditional bulk mixture method, one signal profile may be generated for multiple signals from all of the cells in a mixture containing n cells.

In some embodiments, single-cell methods may combine several steps into an efficient direct-to-PCR extraction and amplification process. Individual cells and/or cell types may be separated by a variety of methods as previously mentioned, as well as visually. Non-limiting examples of DNA extraction protocols may include commercially available products or kits, Arcturus® PicoPure™ DNA extraction (ThermoFisher Scientific), DEPArray™ LysePrep DNA extraction (Menarini Silicon Biosystems), ForensicGEM® Zygem™ (Avantor®) extraction, and DirectPCR Lysis extraction (Viogen Biotech). See, e.g., Sheth et al. Int J Legal Med (2021) https://doi.org/10.1007/s00414-021-02503-4.

The signal output may be produced in any manner using any instruments that provide a detectable signal. In embodiments of the disclosure, the signal profiles illustrate signals that have varying intensities in relation to biomolecular markers (e.g., nucleic acid fragment length). These signals may be generated using any instrumentation that is configured to associate signal intensities with various DNA fragment (or allele) lengths. For example, Illumina NextSeq™ (Illumina), Ion Torrent NGS instruments (e.g., Ion GeneStudio S5™ (ThermoFisher Scientific)), and any other instruments or techniques that generate signals from each cell identifies the DNA fragment length with respect to signal intensity that may be measured by, for example, but not limited to, fluorescence, chemiluminescence, radioactivity, charge, etc. The amplified DNA may be processed to produce such signals for detection, analysis, and subsequent interpretation. Capillary electrophoresis (CE) that produces electropherograms (EPGs) and next-generation sequencing (NGS) (e.g., Illumina (Solexa) sequencing; Roche 454 sequencing; Ion Torrent: Proton/PGM sequencing) are exemplary methods of producing signals having varying signal intensities, which for some methods may produce fluorescent signals as measured by relative fluorescent units (RFUs).

### Sample Analysis and Interpretation

In some embodiments, the systems and methods of the present disclosure solves technical problems in the technology of automated analyses of biological samples by using quantitative means to assign a cluster of cells to a group where the number of groups represents the number of potential contributors to the sample. The likelihood ratio, which compares the probability of the data given a proposed individual contributed versus the probability the individual did not contribute, is determined for each group of cells. In some embodiments, where for n cells, the group number ranges from 1 to n, where n can be, e.g., one or more, two or more, three or more, four or more, five or more, seven or more, ten or more, or other amount of groups or any multiple thereof.

Accordingly, aspects of embodiments of the present disclosure enable technical improvements to DNA sequencing systems and methods by enabling single cell analysis techniques for select groups of cells to provide the efficiency benefits of bulk cell analysis with the precision of single cell analysis to achieve efficient and reliable results. To do so, some embodiments of the present invention include features for: (i) refined laboratory parameters for commercially available single cell bench-top systems and develop standard operating procedures that can be translated into operations with minimal disruption to current forensic workflows; (ii) development of an optimized likelihood ratio interpretation strategy founded on sound statistical principles; (iii) development of efficient, accurate algorithms that can be translated to external laboratories for testing; and (iv) comparison of single-cell match-statistics with state-of-the-art bulk-sample interpretation systems to identify forensic sample classes for which single cell systems are needed, among other improvements and capabilities.

In some embodiments, probabilistic evaluation of complex DNA may often result in likelihood ratios that approach one, rendering little information to update a user. Therefore, some embodiments of the present disclosure include systems and methods enabling one to fully explore DNA from all contributions using a single-cell deconvolution approach. Thus, single-cell technology is designed with an inference framework suitable for testing hypotheses on collections of single cell profiles. Accordingly, in some embodiments, the systems and methods present state-of-the-art front-end mixture de-convolution pipelines by generating single-cell profiles while developing statistically sound single-cell interpretation algorithms for translation into forensic practice. For example, the front-end mixture de-convolution pipelines may generate, e.g., one, two, three, five, seven, ten, twenty, thirty, or more single-cell profiles or any multiple thereof.

The method is based on one that includes separating cells, extracting and amplifying the sample to target loci-of-interest, analyzing each cell to produce a data profile for each cell; proposing a suggested number of cell-groups; and comparing the data profiles from each group to a set of simulated genotypes to give an indication of the likelihood of the cell grouping given the suggested genotype.

In some embodiments, interpreting a collection of signal profile measurements can be approached in at least three ways: (I) by assessing each signal profile measurements in isolation from the others; (II) by clustering, i.e. gathering, signal profiles into groups determined to represent a single genotype for collective, cell-group-based, inference; or (III) by jointly analyzing all the signal profile measurements together, which is similar to, but not the same as, the interpretation of technical replicates. In ideal circumstances, each single-cell would result in a full STR profile. In that case, interpretation is straightforward and could be achieved by binary methods with the forensic DNA analyst grouping the signal profile measurements unambiguously. Due to artifacts such as dropout, stutter and instrument noise, however, signal profiles from the same genetic source must be treated as stochastic objects. If these sources of variability in signal profiles are non-negligible, the first interpretation approach, (I), inherently suffers from family-wise error. That is, as more single cell signal profiles are examined, an incorrect genotype call is increasingly likely to be made due to a random combination of non-genotype sources of signal. The preliminary data explored below indicates that even for relatively pristine data, one cannot expect the simplicity of full, unambiguous, STR profiles from each cell. Consequently, a more holistic interpretation scheme that assesses signal profiles in groups or jointly, along the lines of (II) and (III), is necessary.

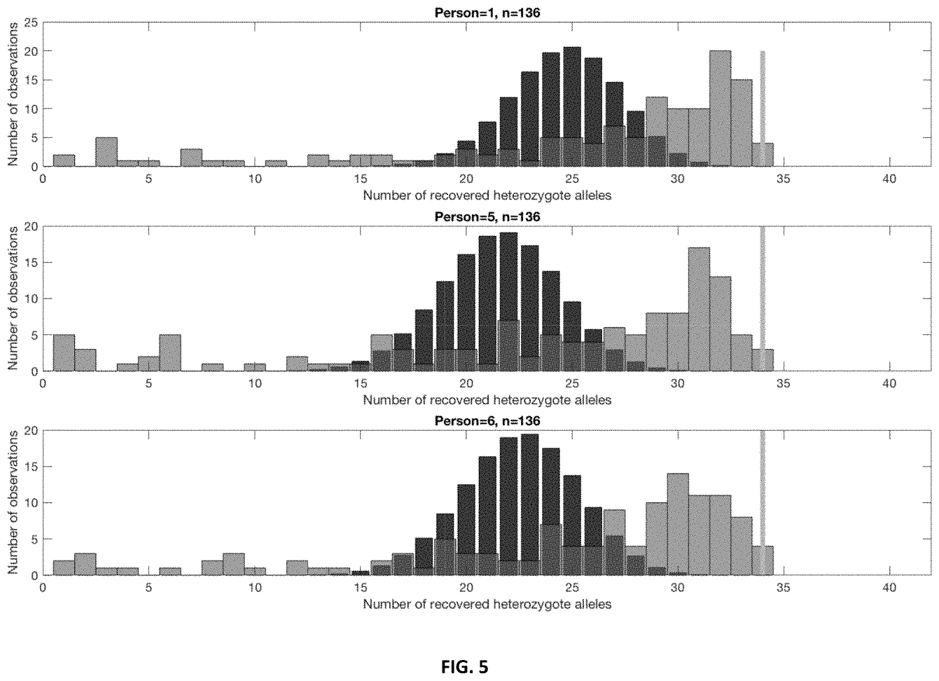

Accordingly, in some embodiments, a step for single cell characterization is employed. Allele Dropout is not cell-independent in the single-cell regime. Using the example data previously described, allelic dropout may be evaluated for samples from three people, Persons 01, 05 and 06, each of who have 34 heterozygous alleles. Thirty-four single cell samples in this example may be analyzed per person for each of four extraction kits, giving a total of 4,624 heterozygous allelic positions per-person. FIG. 5 plots the histogram of the number of alleles observed for each of the 136 signal profile measurements for each person (blue histogram). Most of the profiles rendered ‘good quality’ profiles where at least 75% of the heterozygous alleles were labeled, and the modes of the histograms are located at 32, 31 and 30 alleles for Person 01, 05 and 06, respectively. Only a small fraction of profiles (e.g., 3.7%, 2.9% and 3.7% per person 01, 05 and 06, respectively) resulted in detection of all heterozygote alleles, while many were of low- or moderate-quality as seen by the long left-tail in the blue histogram of FIG. 5, corroborating the findings. If allele dropout were independent, nearly all signal profiles would result in partial profiles as the number of recovered alleles per profile would follow a Binomial distribution on 34 trials. The red histograms in FIG. 5 represent the best-fit Binomial distribution based on the empirical dropout probabilities per-person of 0.28, 0.37 and 0.33 respectively, and are entirely inconsistent with the experimental data. These results demonstrate that allele dropout rates are not cell independent and interpretation strategies that assume allele dropout independence ought not be applied to single-cell data. Instead a carefully constructed interpretation strategy for single-cell data is required.

In some embodiments, aspects of single-cell interpretation can include an analysis of stutter. Stutter can obfuscate DNA signal profile such as an electropherogram (EPG) signal. Stutter has been characterized both from a mechanistic and modeling perspective. Simulation studies based on mathematical models suggest stutter signal within the low-template regime is more prevalent than stutter signal in the high-template regime for two reasons: a single strand slippage early in the PCR can result in the stuttered allele being amplified to a similar extent as the true allele; and instrument noise has a larger effect on these already low-level signals.

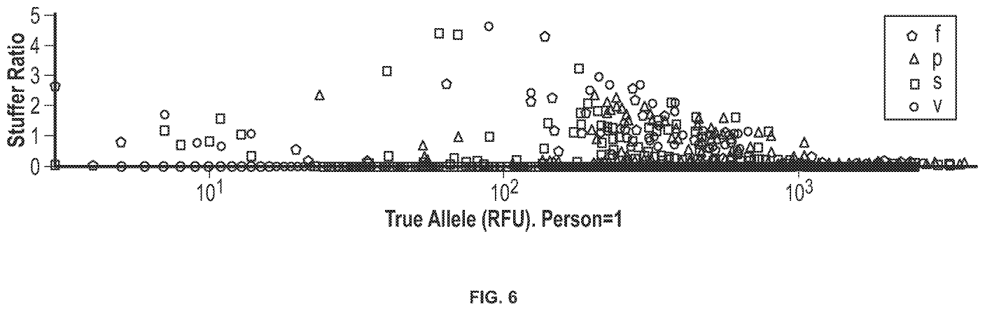

In FIG. 6, Stutter Ratios (SRs) from the single-cell profiles of the example data are plotted against the true allele fluorescence. At relatively large peak heights, e.g., greater than 500, many of the stutter ratios are in excess of 15%. In some embodiments, SRs greater than 15% are within the expected SRs for high copy number samples. For 2.15% of all measurements, the SR is greater than 1, demonstrating that stutter can be a significant confounding factor for single cell signal profiles requiring appropriate consideration during interpretation. Thus, interpretation strategies that are calibrated using high-template samples or do not model stutter as a function of DNA quantity cannot be applied to these data; rather a full-pipeline that takes all pertinent factors into account must be developed.

In some embodiments, taken together the preliminary analysis with the above example data indicates that care must be taken when assessing genotype and match statistics with single cell samples in isolation. Two alternatives are mentioned above, (II) pooling signal profiles into groups determined to be from single contributors and (III) jointly assess all signal profiles. In some embodiments, approach (II) provides a balance that improves both the efficiency and the accuracy of assessing genotype and match statistics, at least relative to the approaches (I) and (III).

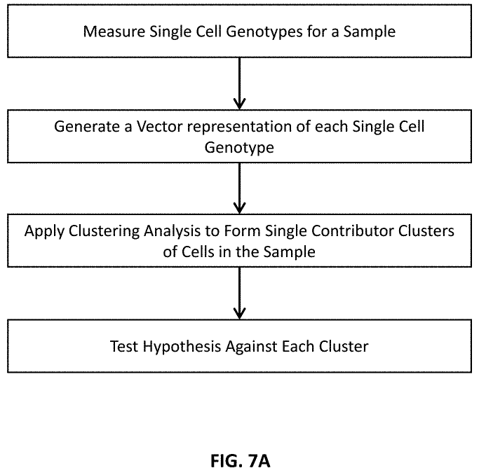

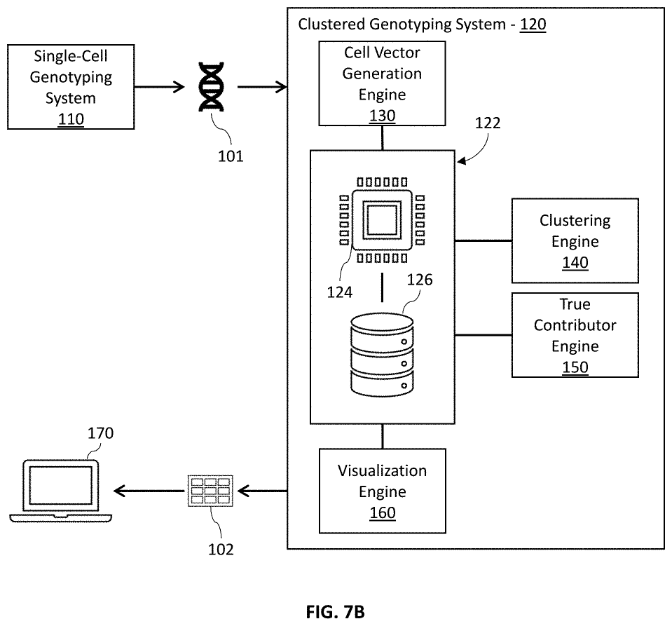

FIG. 7A illustrates a block diagram of an illustrative method for clustered single cell DNA forensics according to embodiments of the present disclosure.

As shown in FIG. 7A, in some embodiments, approach (II) can be implemented according to a four step process for evaluating single cell DNA signal profiles in a sample for assessing genotype and match statistics. The system works by taking groups of profiles of an unknown evidence sample as input along with the allele frequency in the population. The method and system then generate the number of distinct individuals to the cellular admixture while assigning each cell to a specified group. Each group's data is then used to compare the probability of observing the data given an individual contributed versus the probability that they did not.

Testing that true contributors render weights of evidence >1 (favors hypothesis that contributor's DNA is present in sample) reproducibly for at least one group of cells and testing that non-true contributors render weights of evidence <1 for the other groups.

In some embodiments, the four step process can include a step for genotyping single cell DNA sequences for a sample. In some embodiments, the measurement of the DNA sequences can include any suitable DNA signal profile technique. For example, the signal profiles can include, e.g., EPG measurements, current/potential measurements of each locus in a cell, Next Generation Sequencing (NGS), among any other suitable genotyping technology or any combination thereof.

In some embodiments, the signal profiles can be transformed into a vector representation at a second step to enable efficient computer processing and ingestion by a clustering algorithm of the signal profiles. In some embodiments, the vector representation can include, e.g., any suitable vector or set of vectors to describe the genotype of each single cell. In some embodiments, a mapping of a measurement at each locus of each allele in each single cell to an index in a vector for each single cell is employed, which may include a vector for each allele, which each allele vector concatenated together. However, other formats may be employed, such as a vector for each locus with the measurement of that locus from each allele mapped to an index of the vector, and then concatenating each vector together. In some embodiments, the measurements at each locus of each allele of each single cell may be mapped to a respective vector index using the raw measurement, a normalized measurement normalized across the allele or normalized across the cell or normalized across all single cells, or by any other normalization.

In some embodiments, the vectors for each signal profile may be used in a third step to perform clustering of signal profiles. The clustering groups the signal profiles into clusters associated with potential common contributors. For example, a subset of single cells in the sample may originate from a single common contributor. The clustering may implicitly recognize the common contributor and group the signal profiles together due to similarity, likelihood of appear in a common distribution, or according to any other clustering methodology.

In some embodiments, the clusters may be used in a fourth step to test one or more hypotheses against each cluster of cells for, e.g., match statistics, true contributor determination, or other hypothesis. For example, a given target contributor genotype may be tested against each cluster to identify, for each cluster, the probability of a negative hypothesis and a positive hypothesis, where the negative hypothesis includes the assertion that the target contributor does give rise to the cluster, and the negative hypothesis includes the assertion that the target contributor genotype does not give rise to the cluster.

FIG. 7B illustrates a block diagram of an illustrative system for clustered single cell DNA forensics according to embodiments of the present disclosure.

In some embodiments, a clustered genotyping system 120 is utilized with the single-cell genotyping system 110 and at least one computing device 170 to enable the evaluation of clustered signal profiles for assessing genotype and match statistics. In some embodiments, the single-cell genotyping system 110 identifying genotypes of each single-cell in a sample.

In some embodiments, the clustered genotyping system 120 In some embodiments, the clustered genotyping system 120 may be a part of the at least one computing device 170, the single-cell genotyping system 110 or separate computing system. Thus, the clustered genotyping system 120 may include any combination of hardware and/or software components. For example, in some embodiments, the clustered genotyping system 120 may include hardware components including a processing system 122, such as a processor 124, which may include local or remote processing components. In some embodiments, the processor 124 may include any type of data processing capacity, such as a hardware logic circuit, for example an application specific integrated circuit (ASIC) and a programmable logic, or such as a computing device, for example, a microcomputer or microcontroller that include a programmable microprocessor. In some embodiments, the processor 124 may include data-processing capacity provided by the microprocessor. In some embodiments, the microprocessor may include memory, processing, interface resources, controllers, and counters. In some embodiments, the microprocessor may also include one or more programs stored in memory.

Similarly, the processing system 122 may include storage 126, such as local hard-drive, solid-state drive, flash drive, database or other local storage, or remote storage such as a server, mainframe, database or cloud provided storage solution.

In some embodiments, the clustered genotyping system 120 may implement computer engines for producing vectors for signal profiles 101, clustering the signal profiles, assessing the clustered genotypes and match statistics, and generating visualizations of clustering and match statistic results. In some embodiments, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

In some embodiments, the clustered genotyping system 120 may receive signal profiles 101 of a sample from the single-cell genotyping system 110 to analyze each genotype in the sample. In some embodiments, the clustered genotyping system 120 may be in direct or networked communication with the single-cell genotyping system 110. For example, the single-cell genotyping system 110 may provide the signal profiles 101 to the clustered genotyping system 120 via, e.g., one or more suitable data communication protocols/modes such as, without limitation, wireless communication protocols including IPX/SPX, X.25, AX.25, AppleTalk™, TCP/IP (e.g., HTTP), Bluetooth™, near-field wireless communication (NFC), RFID, Narrow Band Internet of Things (NBIOT), 3G, 4G, 5G, GSM, GPRS, WiFi, WiMax, CDMA, satellite, ZigBee, wired communication protocols including universal serial bus (USB), Serial ATA (SATA), Peripheral Component Interconnect Express (PCIe), Ethernet, or other wired communication protocol and other suitable communication modes or any combination thereof.

In some embodiments, the network, wired or wireless, may include any suitable computer network, including, two or more computers that are connected with one another for the purpose of communicating data electronically. In some embodiments, the network may include a suitable network type, such as, e.g., a local-area network (LAN), a wide-area network (WAN) or other suitable type. In some embodiments, a LAN may connect computers and peripheral devices in a physical area, such as a business office, laboratory, or college campus, by means of links (wires, Ethernet cables, fiber optics, wireless such as Wi-Fi, etc.) that transmit data. In some embodiments, a LAN may include two or more personal computers, printers, and high-capacity disk-storage devices called file servers, which enable each computer on the network to access a common set of files. LAN operating system software, which interprets input and instructs networked devices, may enable communication between devices to: share the printers and storage equipment, simultaneously access centrally located processors, data, or programs (instruction sets), and other functionalities. Devices on a LAN may also access other LANs or connect to one or more WANs. In some embodiments, a WAN may connect computers and smaller networks to larger networks over greater geographic areas. A WAN may link the computers by means of cables, optical fibers, or satellites, or other wide-area connection means. In some embodiments, an example of a WAN may include the Internet.

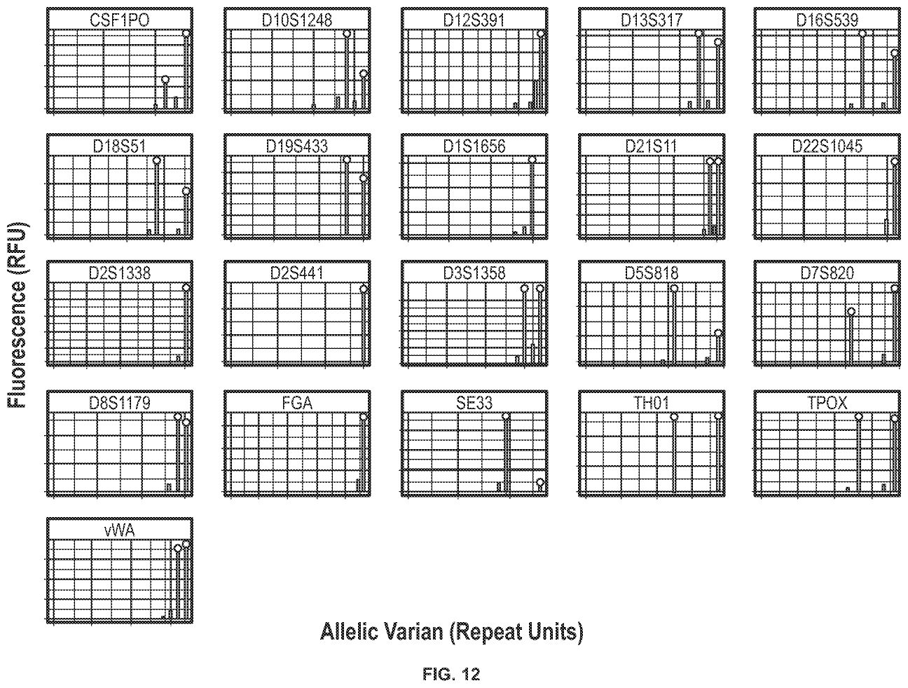

In some embodiments, the single-cell genotyping system 110 may produce any suitable signal profile data. In some embodiments, the single-cell genotyping system 110 may measure presentation single-nucleotide polymorphisms (SNPs) at predetermined loci for each allele of each single-cell. The data for each locus may include, e.g., a locus, an allele and a magnitude according to the measurement technique. For example, the single-cell genotyping system 110 may utilize electrophoresis to produce, for each single-cell a corresponding EPG (see, for example, FIG. 12 below). However, any other type of genotyping technique may be employed, such as, e.g., Next Generation Sequencing (NGS) as described above or any other suitable technique.

In some embodiments, to generate a vector presentation of each signal profile, the clustered genotyping system 120 may utilize a cell vector generation engine 130. In some embodiments, the cell vector generation engine 130 may include dedicated and/or shared software components, hardware components, or a combination thereof. For example, the cell vector generation engine 130 may include a dedicated processor and storage. However, in some embodiments, the cell vector generation engine 130 may share hardware resources, including the processor 124 and storage 126 of the processing system 122.

In some embodiments, the cell vector generation engine 130 may use filter, such as a high pass filter before or after vector creation. In some embodiments, the filter may be used to restrict the use of genotyping measurements that include too few true alleles. In some embodiments, the filter may be a high pass filter that employs, e.g., an intensity of the genotyping measurements or other measure. For example, an intensity can be formulated that the includes the sum of all peak heights record for a signal profile 101. Thus, the intensity can serve as a proxy for a number of alleles recovered for each single-cell, thus indicating a quality of the signal profiles 101, with the lower quality (e.g., below a threshold intensity) filtered out.

In some embodiments, the intensity can be formulated based on a logarithmic transformation to the genotyping measurements of each single-cell, such as, e.g., a base 10 log or other log transformation.

In some embodiments, the set of signal profiles 101, e.g., the set remaining after the high pass filter, or the total set if high pass filtering is omitted, may be transformed into vector form for ingestion by the clustering engine 140. In some embodiments, An EPG can be described by a series of triples, (l, aᵢ, mᵢ), where l is the locus in a set of loci, aᵢ is the allelic variant and mᵢ the corresponding genotyping measurement recorded at aᵢ (e.g., fᵢ for the measure fluorescence at aᵢ or other measurement).

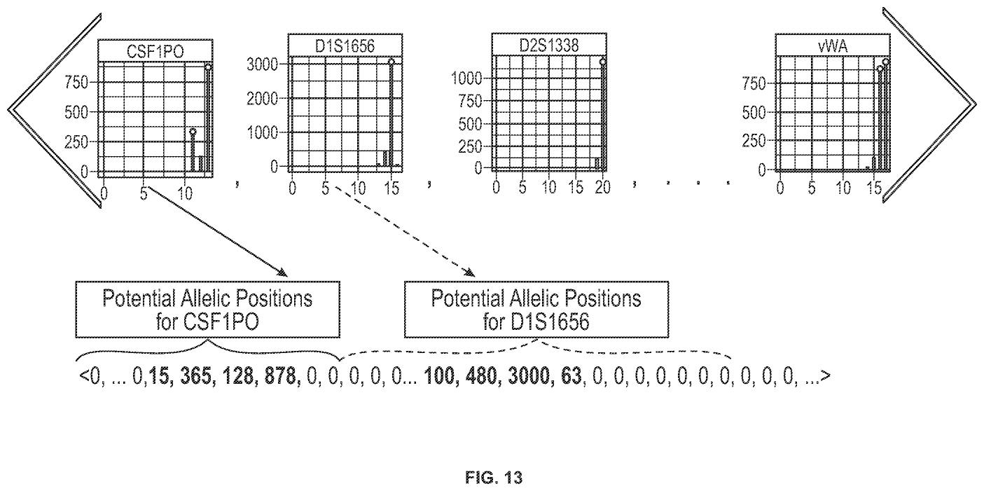

In some embodiments, the genotyping measurement at each locus of a signal profile are treated differently and indeed may be measurements with different mediums, having many different ranges of intensities. In order to make these data comparable it makes sense to embed them in a single high dimensional space. In some embodiments, the cell vector generation engine 130 may embed the measurements in a vector by taking each potential allele location and giving it a unique vector index. The measurement at each allele location (e.g., each locus) may be entered into the corresponding vector index to create a multi-dimensional allele vector for each allele. The allele vectors for a given single-cell may then be concatenated together to form the high dimensional space vector representative of the signal profile 101 for each single-cell (see, for example. FIG. 13). In some embodiments, each allele may be measured at, e.g., 16, 17, 18, 19, 20, 21, 22 or other suitable number of loci. As a result, each signal profile 101 can be represented in a data structure interpretable by software algorithms of, e.g., the clustering engine 140, the visualization engine 160 and/or the true contributor engine 150, among others.

In some embodiments, based on the vector representation of each signal profile 101, the clustered genotyping system 120 may utilize a clustering engine 140 to cluster the signal profiles 101. In some embodiments, the clustering engine 140 may include dedicated and/or shared software components, hardware components, or a combination thereof. For example, the clustering engine 140 may include a dedicated processor and storage. However, in some embodiments, the clustering engine 140 may share hardware resources, including the processor 124 and storage 126 of the processing system 122.

In some embodiments, the clustering engine 140 may utilize any suitable cluster model or algorithm to group signal profile vectors that are likely from a common contributor. In some embodiments, cluster models or algorithms can include, e.g., any unsupervised algorithm including unsupervised machine learning algorithms. In some embodiments, for example, the determine the groupings, any suitable algorithm for determining similarity or probability may be employed, such as, e.g., similarity-based clustering (e.g., centroid models, connectivity models, density models, etc.), distribution models (e.g., expectation maximization algorithms for mixture models, multivariate distribution models including multivariate Gaussian or multivariate normal distribution models), neural network models (e.g., self-organizing maps, etc.), or any other suitable model for clustering multidimensional vectors according to commonalities or any combination thereof.

In some embodiments, after clusters have been formed by an unsupervised machine learning algorithm, they can be refined (i.e. sub-divided further or amalgamated) by assessment of the contents of clusters by a forensics-aware methodology for evaluating the likely number of contributors. If examination of the contents of a cluster suggests it contains more than one genotype, it can be split. Conversely, if n clusters are found, by forming each distinct pair of clusters and assessing the NoC of those pairwise, no more than n(n+1)/2 assessments are necessary to determine what, if any, amalgamation is warranted.

In some embodiments, to analyze match statistics and determine true contributor likelihoods, the clustered genotyping system 120 may employ a true contributor engine 150. In some embodiments, the true contributor engine 150 may include dedicated and/or shared software components, hardware components, or a combination thereof. For example, the true contributor engine 150 may include a dedicated processor and storage. However, in some embodiments, the true contributor engine 150 may share hardware resources, including the processor 124 and storage 126 of the processing system 122.

In some embodiments, the true contributor engine 150 may assess match statistics based on each cluster of signal profiles 101. In some embodiments, within the forensic sciences, the accepted method by which to report the weight of DNA evidence in the courtroom is by presenting Likelihood Ratio (LR), which compares the probability of observing the evidence under two alternative hypotheses, and is expressed as:

$$L R = \frac{P r ( E \vert {H}_{1} , I )}{P r ( E \vert {H}_{2} , I )} , \qquad ( Eq . 1 )$$

where E is the evidence and H1 and H2 are two competing hypotheses, and I is the case or contextual information. The numerator is the probability of observing the evidence given the person of interest is a contributor to the item of evidence (sometimes termed the prosecution's hypothesis, H1 in forensics) and the denominator is the probability of observing the evidence given the person of interest did not contribute to the item of evidence (the defense's hypothesis, H2). The evidence shows support for the prosecution's hypotheses if LR>1, while if LR<1 the defense's hypothesis is supported.

In some embodiments, the clustered genotyping system 120 may employ a visualization engine 160 to provide results, such as, e.g., visualizations of the signal profiles 101, visualizing clusters of signal profiles 101, among other data visualizations. In some embodiments, the visualization engine 160 may include dedicated and/or shared software components, hardware components, or a combination thereof. For example, the visualization engine 160 may include a dedicated processor and storage. However, in some embodiments, the visualization engine 160 may share hardware resources, including the processor 124 and storage 126 of the processing system 122.

In some embodiments, because the signal profiles 101 are represented in vector form as multidimensional vectors in a multidimensional space, the visualization engine 160 may utilize dimensionality reduction to project the signal profile vectors into a renderable format.

In some embodiments, dimensionality reduction may include, e.g., any suitable technique for use in genealogical and genome-wide association studies including Principle Component Analysis (PCA) and Independent Component Analysis (ICA) and modern methods, particularly driven by single-cell RNA sequencing data, such as Uniform Manifold Approximation and Projection (UMAP) and t-Distributed Stochastic Neighbor Embedding (t-SNE). However, in some embodiments, any suitable feature projection may be used to transform the data from the high-dimensional space to a space of fewer dimensions. The data transformation may be linear, as in principal component analysis (PCA), but many nonlinear dimensionality reduction techniques also exist. For multidimensional data, tensor representation can be used in dimensionality reduction through multilinear subspace learning. Other examples may include, e.g., non-negative matrix factorization (NMF), kernel PCA, graph-based kernel PCA, linear discriminant analysis (LDA), generalized discriminant analysis (GDA), autoencoder, etc.

When projecting the signal profile vectors in a low dimensional space, the data may follow a Gaussian distribution resulting in ICA plots that are very similar to the PCA and again t-SNE may have similar results to UMAP. In some embodiments, given the logarithm of the data follows a Gaussian distribution, PCA may be the best with the logarithm of both raw signal and normalized signal. In some embodiments, there may be more information to be gleaned from the PCA than the UMAP, particularly for imbalanced mixtures. There is something to be learned by applying the PCA dimensional reduction techniques on the raw data too as it becomes apparent that the distance from the origin in a PCA plot is a good surrogate for EPG intensity.

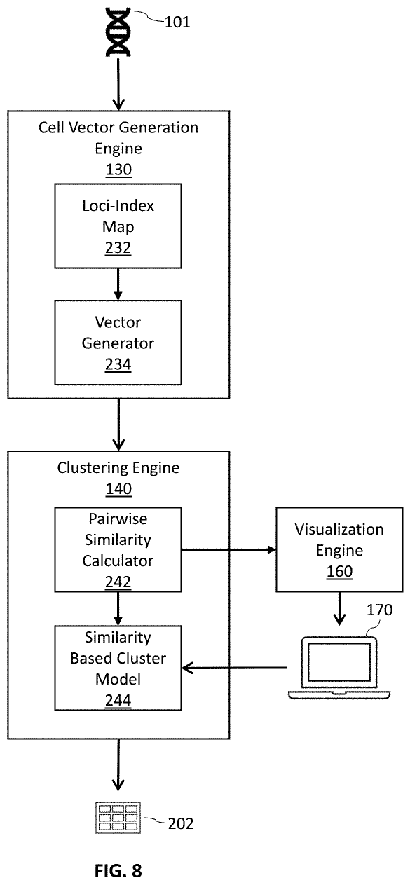

FIG. 8 illustrates a block diagram of an illustrative system for clustering single cell signal profiles for clustered single cell DNA forensics according to embodiments of the present disclosure.

In some embodiments, the genotyping measurement at each locus of a signal profile are treated differently and indeed may be measurements with different mediums, having many different ranges of intensities. In order to make these data comparable it makes sense to embed them in a single high dimensional space. In some embodiments, the cell vector generation engine 130 may embed the measurements in a vector by taking each potential allele location and giving it a unique vector index according to a loci-index map 232 that maps each locus of each allele to a particular index in the vector. The measurement at each allele location (e.g., each locus) may be entered into the corresponding vector index to create a multi-dimensional allele vector for each allele.

In some embodiments, the vector generator 234 may generate a vector from the allele vectors created by the loci-index map 232. In some embodiments the vector generator 234 may create a signal profile vector by concatenating the allele vectors for a given single-cell in a specified order. Thus, the vector generator 234 may output signal profile vectors that represent high dimensional space vectors representative of each signal profile 101.

In some embodiments, the signal profile vectors may be constructed as forensic ignorant vectors such that one vector, Vₖ^{G}, will describe a signal profile 101 in full. G is the genotype ID and k∈{1, . . . , n_{SP}} where n_{SP} is the total number of signal profiles for genotype G. In some embodiments, signal profile vectors may be forensic ignorant because the magnitudes or peaks have been concatenated in such a way that one cannot readily determine at which loci a peak was recorded thus treating a signal profile as a single high dimensional signal. This method can be applied to any signal profile data, but the dimensions may be data specific. In some embodiments, the signal profile vector Vₖ^{G} may be constructed as follows:

Create a zero vector of length m, such that:

m=Σₗ₌₁^{p}nₗ (Eq. 2)

where nₗ is the data specific set of all potential allelic variants for the locus l of a set of loci p, where the set of loci p can include any suitable number of loci (e.g., five, ten, fifteen, twenty, twenty one, twenty two, etc.) such that:

nₗ=4(┌aₘₐₓ^{l}┐−└aₘᵢₙ^{l}┘)+1 (Eq. 3)

where aₘᵢₙ^{l} and aₘₐₓ^{l} are the minimum and maximum allelic variants recorded for locus l across all genotypes in our data. In some embodiments, the allelic variants may include non-integer allelic variants and so to account for this the floor and ceiling of the min and max, respectively, are employed. It is also for this reason that there is multiplier by a factor of 4 and an offset of 1 is employed to ensure the correct number of positions available. In some embodiments, a_{min/max}^{l} across all genotypes present in the data to ensure |Vₖ^{G}| is constant for all G and k. In some embodiments, if the signal is zero for all samples at a given vectorial location, that position is removed from the representation.

In some embodiments, to ensure each vector is comparable the loci are consistently concatenated. The order to concatenate is arbitrary but once selected it remains constant. For example, the order may include: {CSF1PO, D1S1656, D2S1338, D2S441, D3S1358, D5S818, D7S820, D8S1179, D10S1248, D12S391, D13S317, D16S539, D18S51, D19S433, D21S11, D22S1045, FGA, SE33, TH01, TPOX, vWA}.

In some embodiments, the clustering engine 140 may ingest the signal profile vectors to perform clustering according to a suitable cluster model. In some embodiments, the clustering may include, e.g., a similarity based clustering algorithm, such as, e.g., k-nearest neighbor or k-means clustering, or other centroid and other similarity algorithms to form clusters of similar data.

Accordingly, in some embodiments, the clustering engine 140 may employ a pairwise similarity calculator 242 to determine a similarity between each pairwise combination of signal profile vectors. In some embodiments, the measure of similarity may include, e.g., Jaccard similarity, Jaro-Winkler similarity, Cosine similarity, Euclidean similarity, Overlap similarity, Pearson similarity, among other similarity measure or any combination thereof.

In some embodiments, some similarity measures such as Euclidean distance is appropriate for data measured on the same scale, for which magnitudes are comparable. However, in some embodiments, signal profile vectors may, have high values yet originate from different contributors. If a Euclidean distance is chosen, observations with high values may be clustered together and those with low values may thus be clustered incorrectly by incorrectly grouping single-cells by their magnitude rather than their genotype.

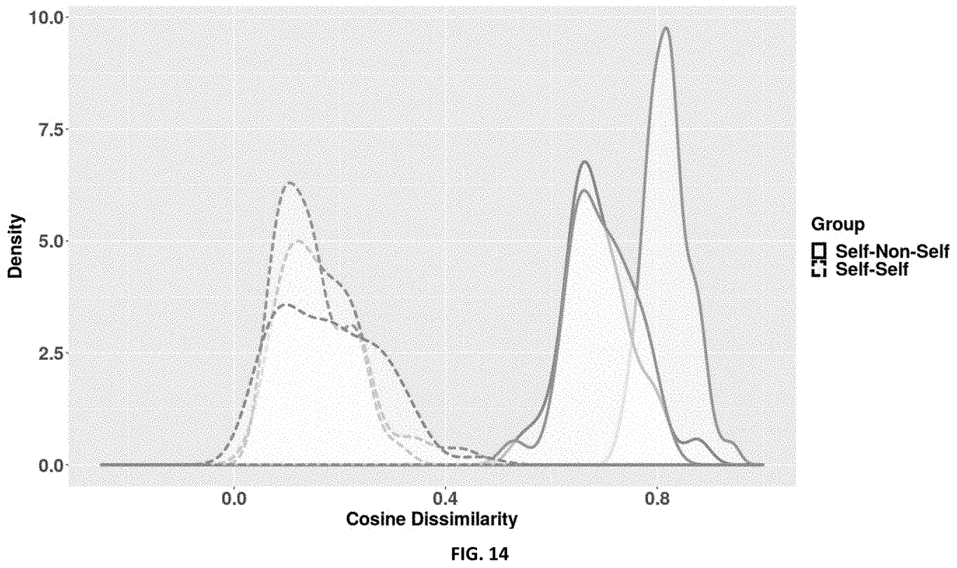

Accordingly, in some embodiments, signal profile vectors may be more accurately assessed for similarity according to overall profiles irrespective of magnitudes. Thus, in some embodiments, a similarity measure that forgoes magnitude may be advantageous. For example, cosine similarity relates observations by measuring the cosine of the angle between two non-zero vectors projected into a n-dimensional space, thus ignoring any reliance on magnitude. Observed values may be far apart in terms of a Euclidean distance but they may have a small angle between them implying high similarity. Vectors with the same orientation have a cosine similarity of 1 while two vectors with a perpendicular orientation have a cosine similarity of 0. In some embodiments, the pairwise similarity calculator 242 may employ a cosine metric based on this logic that equates to saying signal profile vectors originating from the same genotype will lie close to 0 whereas signal profile vectors form different genotypes will lie close to 1 (see, for example, FIG. 14 below).

In some embodiments, to facilitate similarity based clustering, such as with k-mean clustering, a user may select a number of clusters. To allow the user to select the correct number of clusters, the pairwise similarity calculator 242 may output the distribution of pairwise similarities to the visualization engine 160. In some embodiments, the visualization engine 160 may interface with the computing device 170 to depict the cosine similarities or other suitable similarity metrics. Accordingly, using the dimensionality reduction aspects of the visualization engine 160 such as PCA or ICA as described above and as described in further detail below, the clusters according to a cosine similarity metric may be visually apparent on a display of the computing device 170. As a result, the user may select the number of clusters for, e.g., k-means clustering.

In some embodiments, total signal profiles are dominated by true allele peak heights and so to determine which distribution best describes the sample of signal profile vectors, the true allele signal may be utilized. In some embodiments, a vector normalization may be employed to determine a normal and/or a log-normal distribution for the signals represented by each signal profile vector. In some embodiments, these distributions on raw signal and on normalized signal. We have normalized signal profiles as follows:

$$\frac{{f}_{i}^{{SP}_{k}}}{{I}_{k}} \qquad ( Eq . 4 )$$

where fᵢ^{SP}^{ₖ} are the signal profiles recorded for each signal profile vector SPₖ, i, k∈ [symbol] and Iₖ is the intensity of each signal profile vector SPₖ.

In some embodiments, true allele peak heights are best described by log-normal distributions. The log-normal distribution class provides statistical consistency with both the raw-signal and the normalized-signal, where the data is transformed by taking the logarithm to the base 10 and find the best fit normal. In some embodiments, this fit falls closely in line with the data when compared to the best fit normal of raw-signal data. As a result, when using clustering methods such as PCA or mclust, which assume that the data are normally distributed, the vector normalization 342 may take the logarithm base ten of a normalized dataset of signal profile vectors as the input.

In some embodiments, a similarity-based cluster model 244 may receive the similarity metrics, signal profile vectors, the number of clusters. In some embodiments, the similarity-based cluster model 244 may include, e.g., k-means clustering, as described above, however any other suitable similarity based cluster model may be employed, such as, e.g., k-medians, k-medoids, fuzzy c-means, k-means+, kd-trees, or any other suitable clustering analysis or any combination thereof.

In some embodiments, the similarity-based cluster model 244 may utilize the similarity metric assign each signal profile vector to a particular cluster based on the number of clusters selected by the user. As a result, the similarity-based cluster model 244 may output clusters of clustered signal profile vectors 202 having a number of clusters equal to the number selected by the user.

In some embodiments, the user, e.g., via an output by the visualization engine 160 or the similarity-based cluster model 244 may iteratively refine the clusters. For example, the similarity-based cluster model 244 may reassess the similarity of the signal profile vectors within each cluster to determine a likely number of contributors or a degree of similarity or similarity based on the signal profile vectors within each cluster. Where the likely number of contributors exceeds the number of clusters, where the likely number of contributors within a given cluster exceeds one, where the dissimilarity of signal profile vectors within a given cluster exceeds a predetermined threshold, or where the similarity of signal profile vectors within a given cluster falls below a predetermined threshold, the similarity-based cluster model 244 may split one or more clusters to more accurately reflect the likely number of contributors. This refinement process may be iteratively performed a predetermined number of times (e.g., two, three, five, ten, etc.) or until certain criteria are met (e.g., a threshold number of re-clustered signal profile vectors falls below a threshold amount, etc.).

Similarly, for example, where the number of clusters exceeds the likely number of contributors, where the dissimilarity of signal profile vectors within a given cluster falls below a predetermined threshold, where the similarity of signal profile vectors within a given cluster exceeds a predetermined threshold, or where two or more clusters exhibit a similarity (e.g., between signal profile vectors or between statistics representative of each cluster) that exceeds a predetermined threshold, the similarity-based cluster model 244 may combine one or more clusters to more accurately reflect the likely number of contributors. This refinement process may be iteratively performed a predetermined number of times (e.g., two, three, five, ten, etc.) or until certain criteria are met (e.g., a threshold number of re-clustered signal profile vectors falls below a threshold amount, etc.).

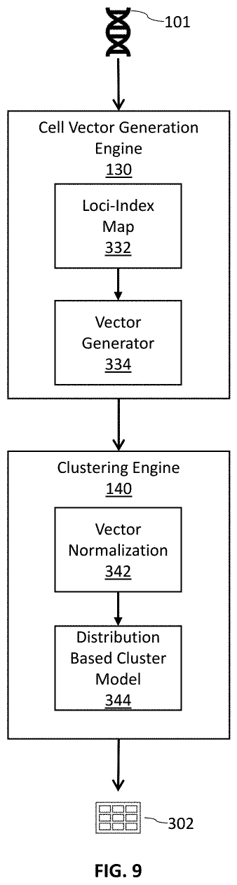

FIG. 9 illustrates a block diagram of another illustrative system for clustering single cell signal profiles for clustered single cell DNA forensics according to embodiments of the present disclosure.

In some embodiments, the genotyping measurement at each locus of a signal profile are treated differently and indeed may be measurements with different mediums, having many different ranges of intensities. In order to make these data comparable it makes sense to embed them in a single high dimensional space. In some embodiments, the cell vector generation engine 130 may embed the measurements in a vector by taking each potential allele location and giving it a unique vector index according to a loci-index map 232 that maps each locus of each allele to a particular index in the vector. The measurement at each allele location (e.g., each locus) may be entered into the corresponding vector index to create a multi-dimensional allele vector for each allele.

In some embodiments, the vector generator 234 may generate a vector from the allele vectors created by the loci-index map 232. In some embodiments the vector generator 234 may create a signal profile vector by concatenating the allele vectors for a given single-cell in a specified order. Thus, the vector generator 234 may output signal profile vectors that represent high dimensional space vectors representative of each signal profile 101.

In some embodiments, the signal profile vectors may be constructed as forensic ignorant vectors such that one vector, Vₖ^{G}, will describe a signal profile 101 in full. G is the genotype ID and k∈{1, . . . , n_{SP}} where n_{SP} is the total number of signal profiles for genotype G. In some embodiments, signal profile vectors may be forensic ignorant because the magnitudes or peaks have been concatenated in such a way that one cannot readily determine at which loci a peak was recorded thus treating a signal profile as a single high dimensional signal. This method can be applied to any signal profile data, but the dimensions may be data specific. In some embodiments, the signal profile vector Vₖ^{G} may be constructed as follows:

Create a zero vector of length m, such that:

m=Σₗ₌₁^{p}nᵢ (Eq. 5)

where nₗ is the data specific set of all potential allelic variants for the locus l of the set of loci p such that:

nₗ=4(┌aₘₐₓ^{l}┐−└aₘᵢₙ^{l}┘)+1 (Eq. 6)

where aₘᵢₙ^{l} and aₘₐₓ^{l} are the minimum and maximum allelic variants recorded for locus l across all genotypes in our data. In some embodiments, the allelic variants may include non-integer allelic variants and so to account for this the floor and ceiling of the min and max, respectively, are employed. It is also for this reason that there is multiplier by a factor of 4 and an offset of 1 is employed to ensure the correct number of positions available. In some embodiments, a_{min/max}^{l} across all genotypes present in the data to ensure |Vₖ^{G}| is constant for all G and k. In some embodiments, if the signal is zero for all samples at a given vectorial location, that position is removed from the representation.

In some embodiments, to ensure each vector is comparable the loci are consistently concatenated. The order to concatenate is arbitrary but once selected it remains constant. For example, the order may include: {CSF1PO, D1S1656, D2S1338, D2S441, D3S1358, D5S818, D7S820, D8S1179, D10S1248, D12S391, D13S317, D16S539, D18S51, D19S433, D21S11, D22S1045, FGA, SE33, TH01, TPOX, vWA}.

In some embodiments, the clustering engine 140 may ingest the signal profile vectors to perform clustering according to a suitable cluster model. In some embodiments, the cluster model may include a distribution-based cluster model 344. Accordingly, in some embodiments, a distribution-based cluster model 344 utilizing distributions matching distributions of the sampled data, e.g., the signal profile vectors.

In some embodiments, total signal profiles are dominated by true allele peak heights and so to determine which distribution best describes the sample of signal profile vectors, the true allele signal may be utilized. In some embodiments, a vector normalization 342 may be employed to determine a normal and/or a log-normal distribution for the signals represented by each signal profile vector. In some embodiments, these distributions on raw signal and on normalized signal as set forth with Eq. 4 above where fᵢ^{SP}^{ₖ} are the signal profiles recorded for each signal profile vector SPₖ, i, k∈ [symbol] and Iₖ is the intensity of each signal profile vector GMₖ.

In some embodiments, true allele peak heights are best described by log-normal distributions. The log-normal distribution class provides statistical consistency with both the raw-signal and the normalized-signal, where the data is transformed by taking the logarithm to the base 10 and find the best fit normal. In some embodiments, this fit falls closely in line with the data when compared to the best fit normal of raw-signal data. As a result, when using clustering methods such as PCA or mclust, which assume that the data are normally distributed, the vector normalization 342 may take the logarithm base ten of a normalized dataset of signal profile vectors as the input.

In some embodiments, the distribution-based cluster model 344 may include a model that does not require input from the user by both determining the number of clusters along with cluster assignment. In some embodiments, the distribution-based cluster model 344 may include one or more Bayesian methods that determine an A Posteriori Probability on n (“APP(n)”), which may provide powerful tools since such methods can incorporate information on peak heights (including degradation and differential degradation), forward and reverse stutter, noise, and allelic drop-out, while being cognizant of allele frequencies in a reference population. In some embodiments, finite mixture models and model-based clustering, also known as Mixture Models (MM), include a broad family of algorithms designed for modelling an unknown distribution as a mixture of distributions. The probability distribution of observed data is approximated by a statistical model and cluster analysis is performed by estimating the model parameters from the data where the parameters define clusters of similar observations.

In some embodiments, as described above, upon normalizing the sample of signal profile vectors, the distribution may fit a normal distribution. Accordingly, in some embodiments, a mixture model may be used which considers the data as coming from a distribution that is mixture of two or more Gaussian distributions. In some embodiments, using a mixture model with a mixture of Gaussian distributions, the distribution-based cluster model 344 may model each component k by the Gaussian distribution, characterized by a mean vector, pₖ, a covariance matrix, Eₖ and an associated probability in the mixture where each signal profile vector has a probability of belonging to each cluster.

In some embodiments, these parameters are estimated using the expectation-maximization (EM) algorithm and each cluster k is centered at pₖ, with increased density for points near the mean. The geometric features of each cluster, the shape, volume, and orientation, are determined by Eₖ. Functions for performing single Expectation and Maximization steps and for simulating data for each available model are also included. Additional ways of displaying and visualizing fitted models along with clustering, classification, and density estimation results are also contemplated, including neural network modeling, machine learning classification, and optimization algorithms, such as, e.g., Expectation conditional maximization (ECM), Expectation conditional maximization either (ECME), Majorize/Minimize or Minorize/Maximize (MM), factorized Q approximation, moment based algorithms, spectral algorithms, among others or any combination thereof.

In some embodiments, in practice, the distribution-based cluster model 344 may be implemented using a clustering algorithm package of the programming language used to build the clustering engine 140. In some embodiments, the clustering engine 140 may be implemented using R, and the clustering package may include the mclust R package for model-based clustering, classification, and density estimation based on finite Gaussian mixture modelling.

In some embodiments, mclust assumes the data follows a Gaussian distribution. Accordingly, the log-normal signal profile vector distribution described above may outperform alternative transformations and the raw data, such as the log of the raw data.

In some embodiments, using mclust or other suitable clustering package, only the data matrix was provided for function calls. In some embodiments, the number of mixing components may include up to 9, up to 10, up to 11 or more by default and the covariance parameterization are selected using the default Bayesian Information Criterion (BIC). Information criteria are based on penalized forms of the log-likelihood. As the likelihood increases with the addition of more components, a penalty term for the number of estimated parameters is subtracted from the log-likelihood. In some embodiments, a distribution-based cluster model 344 having a four-component mixture with covariances having spherical distributions with the unequal shape and volume or spherical distribution with equal shape and volume may be most likely.

In some embodiments, based on the analysis by the distribution-based cluster model 344, the normalized vectors may be assigned to a most likely distribution and clustered according to the assigned distributions. As a result, the distribution-based cluster model 344 may output clusters of clustered signal profile vectors 302.

In some embodiments, distribution-based cluster model 344 may iteratively refine the clusters. For example, the distribution-based cluster model 344 may reassess the probabilities of the signal profile vectors with respect to the distributions of each cluster to determine a likely number of contributors. Where the likely number of contributors exceeds the number of clusters, where the likely number of contributors within a given cluster exceeds one, where the distribution of signal profile vectors within a given cluster has a probability that exceeds a predetermined threshold, or where the similarity of signal profile vectors within a given cluster has a probability that falls below a predetermined threshold, the similarity-based cluster model 244 may split one or more clusters to more accurately reflect the likely number of contributors. This refinement process may be iteratively performed a predetermined number of times (e.g., two, three, five, ten, etc.) or until certain criteria are met (e.g., a threshold number of re-clustered signal profile vectors falls below a threshold amount, etc.).

Similarly, for example, where the number of clusters exceeds the likely number of contributors, where the likely number of contributors within a given cluster falls below one, where the distribution of signal profile vectors within a given cluster has a probability that falls below a predetermined threshold, or where the distribution of signal profile vectors between multiple clusters has a probability that exceeds a predetermined threshold, the distribution-based cluster model 344 may combine one or more clusters to more accurately reflect the likely number of contributors. This refinement process may be iteratively performed a predetermined number of times (e.g., two, three, five, ten, etc.) or until certain criteria are met (e.g., a threshold number of re-clustered signal profile vectors falls below a threshold amount, etc.).

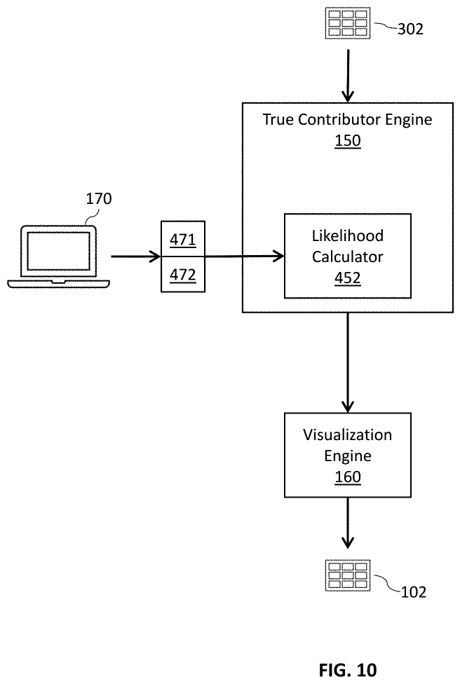

FIG. 10 illustrates a block diagram of an illustrative system for testing DNA sequence hypotheses against clustered single cell signal profiles for clustered single cell DNA forensics according to embodiments of the present disclosure.

The clusters of clustered signal profile vectors 302 output by the pipeline described above is the determination of the NoC and groupings of single cell samples by contributor. For each group, one can then perform single contributor comparisons based on those samples with any existing match statistic methodology. In some embodiments, the match statistic may include the likelihood ratio (LR), which is the generally accepted standard for probabilistic interpretation systems. In some embodiments, the true contributor engine 150 may utilize a likelihood calculator 452 that employs either the average clustered signal per contributor as well as considering each sample, separately. More concretely, suppose that in a particular cluster there are n clusters of clustered signal profile vectors 302, E₁, E₂, . . . , Eₙ, where each EPG Eᵢ is a vector of peak heights. From these EPGs, we generate an average genotype Ê=Σᵢ₊₁ⁿEᵢ/n. Two specific match statistics we will consider are LR_{avg} and LRₛₑₚ, where

$${LR}_{a v g} = \frac{P ( \hat{E} \vert {H}_{1} )}{P ( \hat{E} \vert {H}_{2} )} and \qquad ( Eq . 7 )$$

$$\begin{matrix}L {R}_{s e p} = \frac{P ( {E}_{1} , {E}_{2} , … , {E}_{n} \vert {H}_{1} )}{P ( {E}_{1} , {E}_{2} , … , {E}_{n} \vert {H}_{2} )} & ( Eq . 8 )\end{matrix}$$

Here, H₁ 471 and H₂ 472 refer to the prosecution and defense hypotheses, specifically, which are generally assumed to be that the evidence (e.g. the EPGs) arises from the genotype of a specific target individual for H₁ and that the evidence arises from the genotype of a random individual from the background population. In some embodiments, H₁ 471 and H₂ 472 may be provided for the target individual by, e.g., a user at the computing device 170. In some embodiments, the likelihood calculator 452 may employ signal models similar to those described in Swaminathan, H., Garg, A., Grgicak, C. M., Medard, M. & Lun, D. S. CEESIt: A computational tool for the interpretation of STR mixtures. Forensic Science International-Genetics 22, 149-160 (2016), which is herein incorporated by reference in its entirety.

In some embodiments, each locus may be treated as being probabilistically independent, to describe a model for a full signal profile (“SP”) it is sufficient to restrict attention to describing a model for a single SP (single locus l). Accordingly, in some embodiments, a model that only incorporates the key features: true allele signal, noise and reverse stutter may be employed.

True allele signal is the amount of fluorescence in RFU that comes as a result of detecting a true allelic variant during the process of electrophoresis. There exists insufficient characterization of the true distribution of the random variable A, with declaring it cannot be easily described by a simple distribution class. The gamma distribution has been adopted as it gives a simple yet flexible class of unimodal and asymmetric densities that best fit their simulated data, however it has been suggested by that one could determine the distribution directly when one has sufficient data to do so as it can vary with the quantity of DNA present.

Different loci have a different range of potential alleles and we will define the set of potential alleles for a given locus l, as B^{l}. We will establish a toy model GM), that describes the signal recorded at allele j∈B^{l}, for locus l as follows:

SPⱼ^{l}=Nⱼ+Z₁1_{A}_{₁}_{^{l}}₌ⱼ+Z₂1_{A}_{₂}_{^{l}}₌ⱼ=j+λZ₁1_{A}_{₁}_{^{l}}₌ⱼ₋₁+λZ₂1_{A}_{₂}_{^{l}}₌ⱼ₋₁ (Eq. 9)

where Nⱼ is the noise at allele j. In this model the occurrence of noise can be determined by a binomial distribution. Z₁ and Z₂ are the magnitude of measurements recorded at true allelic variants. In some embodiments, it is assumed that Z follows a log-normal distribution as it appears to reasonably describe the data, as described above. A₁^{l} and A₂^{l} are the true alleles for a given locus, λ is the stutter ratio and 1 is the indicator function.

In some embodiments, this simple model can be used to determine the probability of a signal profile given a genotype, P(SP^{l}|Aᵢ₁^{l}=aᵢ₁^{l},Aᵢ₂^{l})=aᵢ₂^{l}=P(SP^{l}|Gᵢ). However, the probability of the signal profile may be given a genotype, P(SP|Gᵢ):

P(GM|Gᵢ)=Π_{l∈L}P(SP^{l}|Gᵢ^{l}) (Eq. 10)

where L is the set of all loci studied in a forensic DNA profile. L can be determined from CODIS or similar.

In some embodiments, the prosecution's hypothesis calculation may include, e.g., the probability of seeing the cluster of clustered signal profile vectors 302 given the genotype is that of the target individual. Henceforth, the genotype of a person-of-interest (POI) shall be referred to as s. This yields:

P(E|H₁)=Σ_{g}P(SP|G=s)P(G=s|H₁) (Eq. 11)

If the genotype corresponds to a target individual, then A₁^{l} and A₂^{l} become fixed and there exists a genotype s such that:

P(E|H₂)=P(GM|G=s)=Π_{lEL}P(SP^{l}|A₁^{l}=s₁^{l},A₂^{l}+s₂^{l}) (Eq. 12)

In some embodiments, the defense's hypothesis calculation may include the probability that any other individual as the target individual could be responsible for the cluster of clustered signal profile vectors 302.

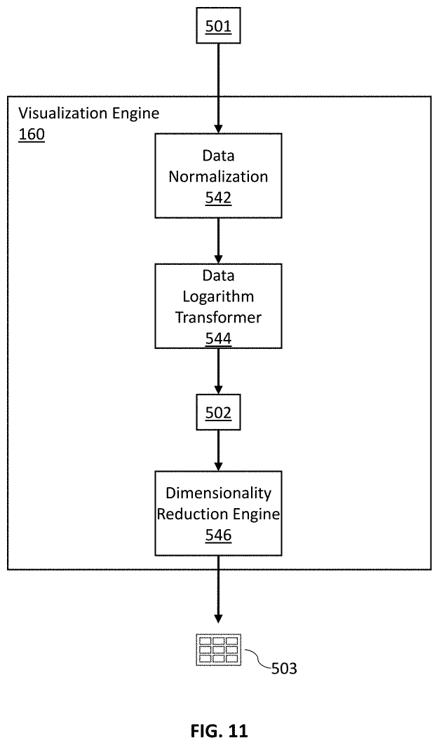

FIG. 11 illustrates a block diagram of an illustrative visualization engine for visualizing clustered single cell DNA forensics according to embodiments of the present disclosure.

As described above, when working with multidimensional data, to visualize the data in a meaningful way, converting the data to a low dimensional form. In some embodiments, the visualization engine 160 may utilize one or more dimensionality reduction techniques, such as, e.g., PCA, ICA, UMAP, t-SNE, among others or any combination thereof.

In some embodiments, to increase the effectiveness of the dimensionality reduction can be improved by normalizing the data to be visualized. Accordingly, in some embodiments, upon receiving multidimensional data 501, such as, e.g., the clustered signal profile vectors 202 and 302, the similarity distribution, the signal profile vectors, or any combination thereof, a data normalization 542 may be utilized to normalize the data.

In some embodiments, the data normalization 542 may normalize data by eliminating the units of measurement, enabling more easy comparison of data. In some embodiments, the data normalization 542 may normalize the data by rescaling to values between 0 and 1, such as by transforming each signal profile vector to have a length of one.

In some embodiments, the normalized data may be transformed by a data logarithm transformer 544 to transform the normalized data using, e.g., a base 10 logarithm, or other suitable base. In some embodiments, the logarithm of the normalized data may result in log-normalized data 502 having a similar distribution to a Gaussian distribution, and thus can be approximated as a Gaussian distribution. Accordingly, dimensionality reduction for Gaussian distributions of high dimension data can be employed to visualize the log-normalized data 502.

In some embodiments, a dimensionality reduction engine 546 may ingest the log-normalized data 502 and apply a dimensionality reduction algorithm. As described above, any suitable dimensionality reduction algorithm or model may be employed. In some embodiments, due to the approximate Gaussian distribution of the log-normalized data 502, the dimensionality reduction engine 546 may employ, for example and without limitation, PCA and/or UMAP. While other dimensionality reduction techniques may be employed, PCA and UMAP provide illustrations of the dimensionality reduction engine 546 utilizing a more traditional linear dimensionality reduction technique and non-linear dimensionality reduction technique.

In some embodiments, PCA identifies a new basis, one that is orthogonal, on which to represent the original data. The new coordinate system is determined sequentially such that the first dimension or Principle Component (PC) describes the greatest variance in the data, the second PC is computed with the constraints of being orthogonal to the first PC and describes the second greatest variance in the data and so on. These new variables are found as uncorrelated linear combinations of the original data set and so, to retain as much of the original variance as possible, it reduces to either solving an eigenvalue/eigenvector problem or, alternatively obtaining the Singular Value Decomposition (SVD) of the (centered) data matrix.

In some embodiments, PCA may assume the mean and variance are sufficient statistics to entirely describe the probability distribution of the log-normalized data 502 and the only zero-mean probability distribution that is fully described by the variance is the Gaussian distribution.

In some embodiments, the number of PCs returned equates to the rank, r, of the original data matrix where in general, the rank of an m×n matrix is r≤min {m, n} or r≤min {m−1, n} for column-centered matrices. Genomic data frequently presents datasets where there are fewer individuals than variables hence, the number of individuals often dictates the rank r.

In some embodiments, to increase efficiency by using a limited number of principal components, each admixture or sample of log-normalized data 502 can be represented by relatively fewer variables instead of thousands. Admixtures can then be explored graphically on a PCA plot of the individuals, making it possible to visually assess similarities and differences between observations.

In some embodiments, the UMAP illustration may construct a high dimensional graph representation of the data, then it optimize a low dimensional graph to be as structurally similar as possible. In some embodiments, unlike PCA:
1) UMAP does not make any assumption about the distribution of the data, so there is no need to transform, and
2) UMAP does not have a straight forward interpretation of distance once projected into a low-dimensional space.

This second point is due to the fact that the UMAP algorithm focus on preserving neighborhood topology rather than absolute distance.

In some embodiments, the dimensionality reduction engine 346 may apply UMAP to a data sample. Because of point 1 above, the data may be the multidimensional data 501 before normalization or log transformation or may use normalized but not transformed data. In some embodiments, UMAP may be implemented using a similarity measure such as any of those described above. In some embodiments, a cosine metric, similar to above, may be used.

The number of approximate nearest neighbors used to construct the initial high dimensional graph corresponds to the n neighbor parameter, it effectively controls how UMAP balances local and global structures. Low values will push more focus on the local structure while higher values will push the focus to the global structure. The default for n neighbors is 15. The min dist parameter controls how tightly UMAP \clumps” points together in the low dimensional graph with low values yielding tightly packed clusters and high values, looser clusters [10] with a default of 0:1.

In some embodiments, upon application of the dimensionality reduction technique or combination of techniques, such as PCA and/or UMAP as described above, or any other technique, the dimensionality reduction engine 346 may output a data plot 503. In some embodiments, the data plot 503 may represent the multidimensional data 501 in a low dimension space, such as, e.g., a two dimensional space or a three dimensional space for effective display by a display device such any suitable two dimensional or three dimensional display. For example, the display may include, e.g., a computer screen, television screen, monitor display, virtual reality display, augmented reality display, three-dimensional display panel, etc.

FIG. 12 illustrates allele fluorescent measurements from electropherogram (EPG) of a single-cell according to aspects of embodiments of the present disclosure.

FIG. 13 illustrates the mapping and conversion of allele fluorescent measurements into a concatenated vector, e.g., using a loci-index map as described above according to aspects of embodiments of the present disclosure. In some embodiments, each allele is designated a location in an order of allele-specific vector segment of indices. Each index within each allele-specific vector segment is assigned a specific locus of the allele. Measurements from each locus are then transferred into the corresponding index of the corresponding allele-specific vector segment. All allele-specific vector segments for cell are concatenated together into a highly multidimensional vector. In some embodiments, each allele may be measured at, e.g., 16, 17, 18, 19, 20, 21, 22 or other suitable number of loci.

FIG. 14 illustrates an example distribution of similarity or dissimilarity according to cosine distances between vectors of signal profiles where the dotted lines indicate self-self dissimilarity and the solid lines indicate self-non-self dissimilarity according to aspects of embodiments of the present disclosure.

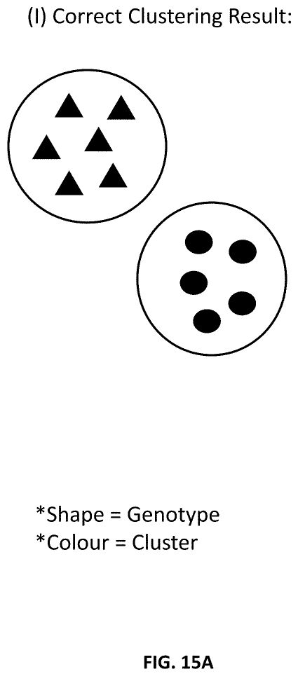

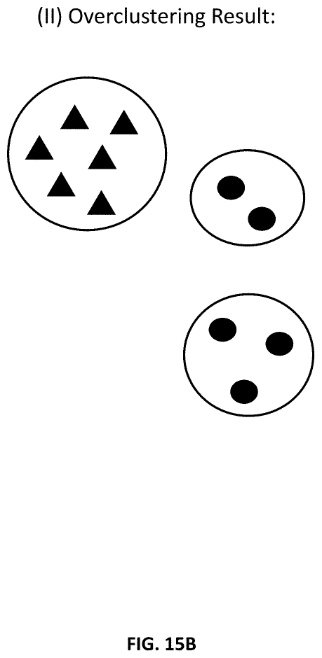

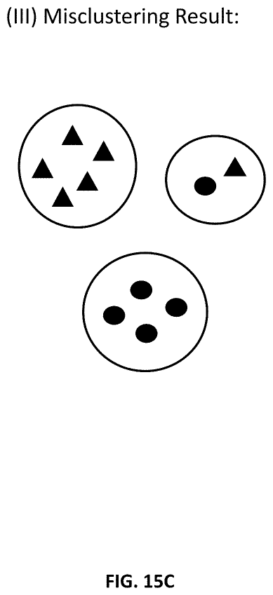

FIG. 15A depicts example illustration of a correct clustering result according to aspects of embodiments of the present disclosure.

FIG. 15B depicts example illustration of an overclustering result according to aspects of embodiments of the present disclosure. In some embodiments, over-clustering may include a situation where a single genotype has been grouped into two or more distinct clusters.

FIG. 15C depicts example illustration of a misclustering result according to aspects of embodiments of the present disclosure. In some embodiments, misclustering as an incident were two or more distinct genotypes are found in one cluster. Misclustering may be of greater concern than overclustering as this can lead to an incorrect description of a genotype. If signal profiles from two (or more) distinct genotypes are clustered together, this may lead to lower likelihood ratios when the POI is a true contributor or larger likelihood ratios when the POI is not a true contributor.

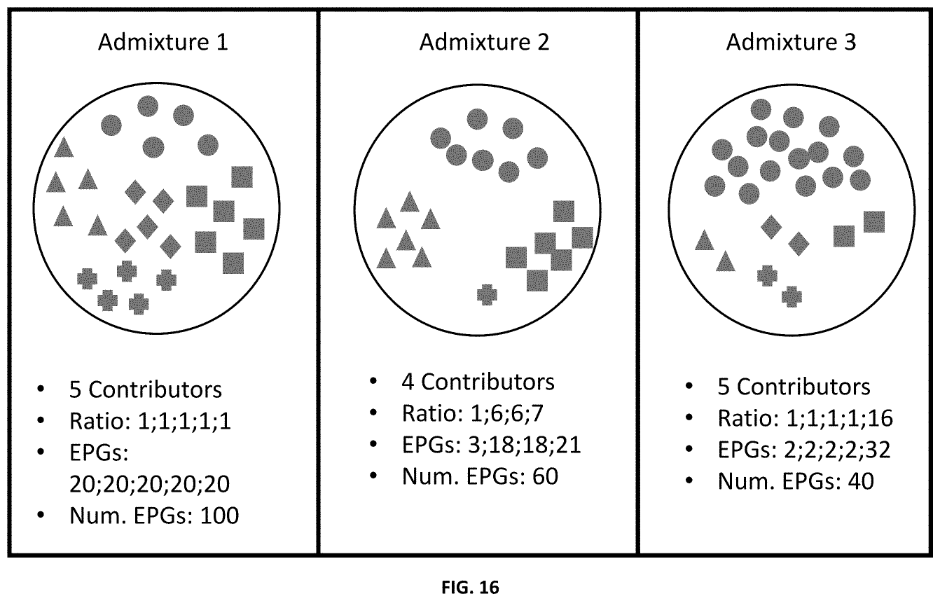

FIG. 16 depicts an example illustration of admixtures having multiple clustered contributors according to aspects of embodiments of the present disclosure. In some embodiments, the admixtures include distribution-based cluster model results for a log-normalized set of signal profiles. Tables 1-3 below indicate the errors for each of Admixture 1, Admixture 2 and Admixture 3 of FIG. 16.

TABLE 1
Percent of Correct Cluster Assignments

| Admixture | % of Correct Cluster Assignments |
|---|---|
| 1 (20; 20; 20; 20; 20) | 98.00% (96.00, 99.33)% |
| 2 (3; 18; 18; 21) | 87.67% (83.67, 91.33)% |
| 3 (2; 2; 2; 2; 32) | 63.67% (58.00, 69.00)% |

TABLE 2
Percent Overclustering

| Admixture | % Overclustering |
|---|---|
| 1 (20; 20; 20; 20; 20) | 2.00% (0.33, 3.67)% |
| 2 (3; 18; 18; 21) | 12.33% (8.33, 16.00)% |
| 3 (2; 2; 2; 2; 32) | 34.33% (28.67, 39.67)% |

TABLE 3
Percent Misclustering

| Admixture | % Misclustering |
|---|---|
| 1 (20; 20; 20; 20; 20) | 0.00% (0.00, 0.33)% |
| 2 (3; 18; 18; 21) | 0.33% (0.00, 1.00)% |
| 3 (2; 2; 2; 2; 32) | 29.67% (24.33, 35.00)% |

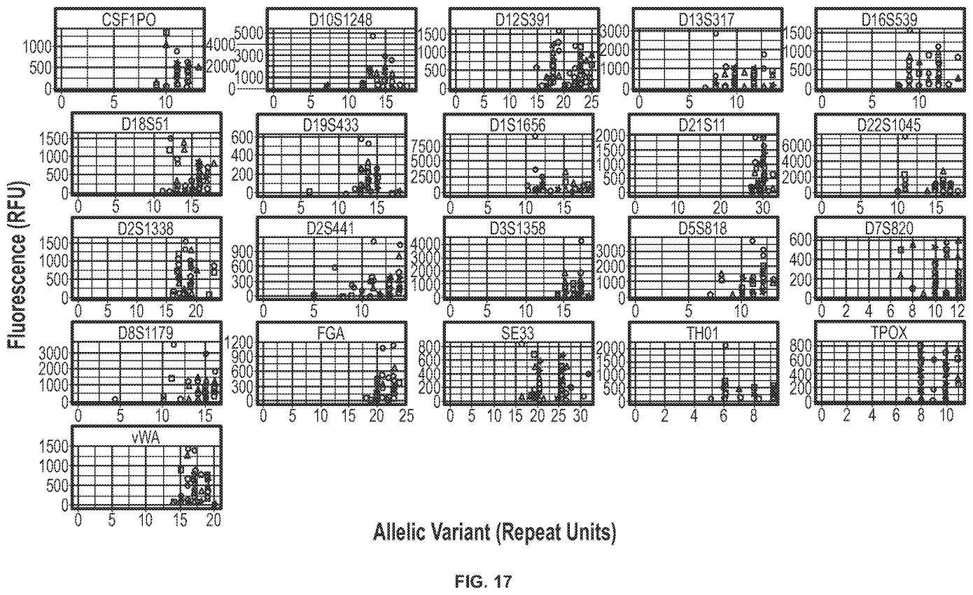

FIG. 17 illustrates an overview of allele signals for a (2;2;2;2;32) simulated admixture according to aspects of embodiments of the present disclosure.

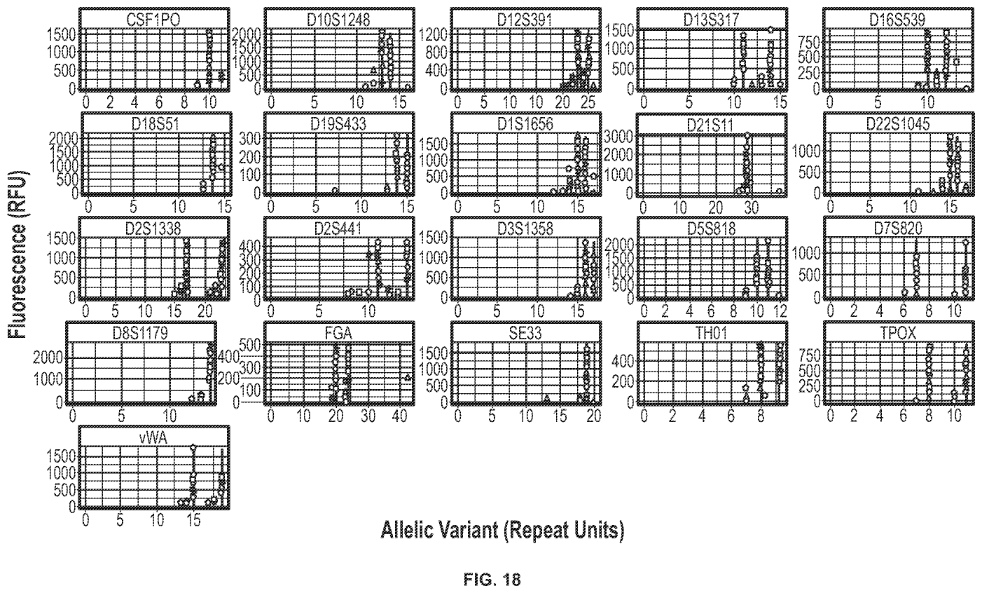

FIG. 18 illustrates an Mclust cluster 5 according to aspects of embodiments of the present disclosure. In some embodiments, the cluster 5 shows that 32 EGS form genotype 02 according to aspects of embodiments of the present disclosure.

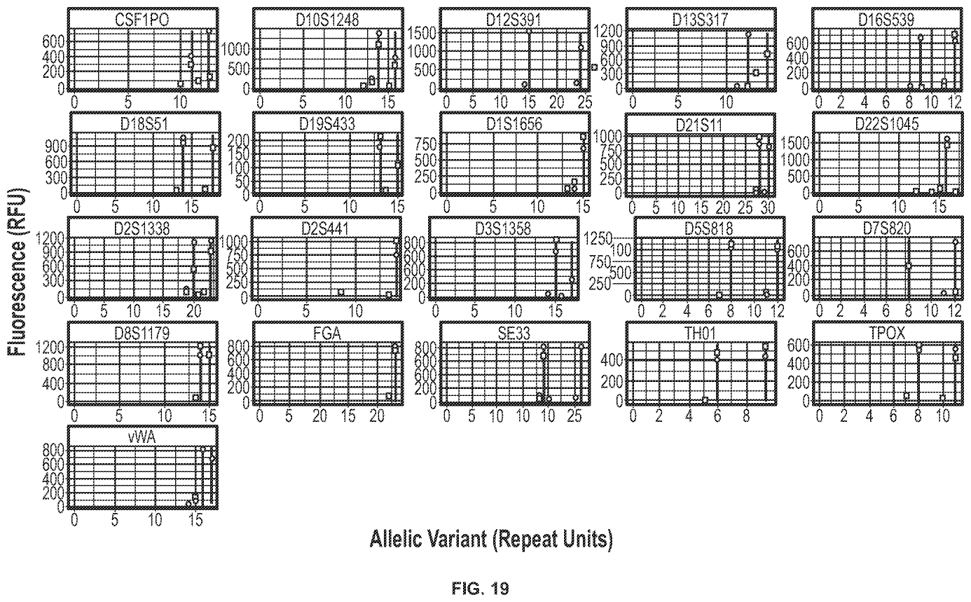

FIG. 19 illustrates an Mclust cluster 1 according to aspects of embodiments of the present disclosure. In some embodiments, the cluster 1 shows that 2 EGS form genotype 06 according to aspects of embodiments of the present disclosure.

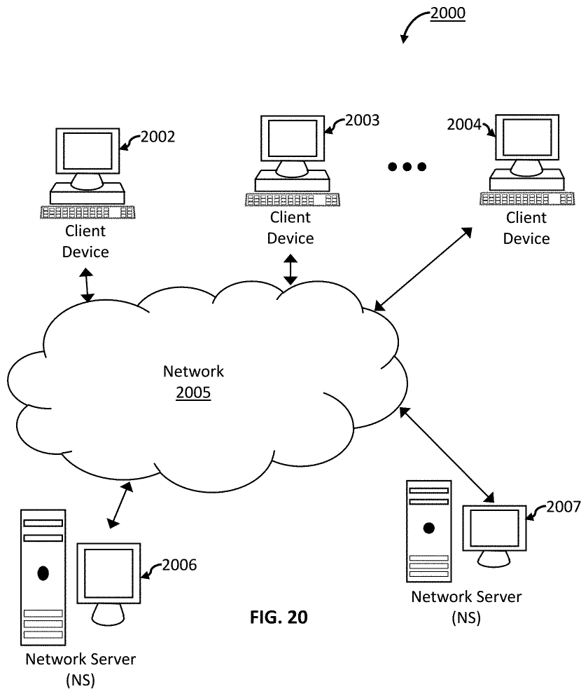

FIG. 20 depicts a block diagram of an exemplary computer-based system and platform 2000 in accordance with one or more embodiments of the present disclosure. However, not all of these components may be required to practice one or more embodiments, and variations in the arrangement and type of the components may be made without departing from the spirit or scope of various embodiments of the present disclosure. In some embodiments, the illustrative computing devices and the illustrative computing components of the exemplary computer-based system and platform 2000 may be configured to manage a large number of members and concurrent transactions, as detailed herein. In some embodiments, the exemplary computer-based system and platform 2000 may be based on a scalable computer and network architecture that incorporates varies strategies for assessing the data, caching, searching, and/or database connection pooling. An example of the scalable architecture is an architecture that is capable of operating multiple servers.

In some embodiments, referring to FIG. 20, members 2002-2004 (e.g., clients) of the exemplary computer-based system and platform 2000 may include virtually any computing device capable of receiving and sending a message over a network (e.g., cloud network), such as network 2005, to and from another computing device, such as servers 2006 and 2007, each other, and the like. In some embodiments, the member devices 2002-2004 may be personal computers, multiprocessor systems, microprocessor-based or programmable consumer electronics, network PCs, and the like. In some embodiments, one or more member devices within member devices 2002-2004 may include computing devices that typically connect using a wireless communications medium such as cell phones, smart phones, pagers, walkie talkies, radio frequency (RF) devices, infrared (IR) devices, CBs, integrated devices combining one or more of the preceding devices, or virtually any mobile computing device, and the like. In some embodiments, one or more member devices within member devices 2002-2004 may be devices that are capable of connecting using a wired or wireless communication medium such as a PDA, POCKET PC, wearable computer, a laptop, tablet, desktop computer, a netbook, a video game device, a pager, a smart phone, an ultra-mobile personal computer (UMPC), and/or any other device that is equipped to communicate over a wired and/or wireless communication medium (e.g., NFC, RFID, NBIOT, 3G, 4G, 5G, GSM, GPRS, WiFi, WiMax, CDMA, satellite, ZigBee, etc.). In some embodiments, one or more member devices within member devices 2002-2004 may include may run one or more applications, such as Internet browsers, mobile applications, voice calls, video games, videoconferencing, and email, among others. In some embodiments, one or more member devices within member devices 2002-2004 may be configured to receive and to send web pages, and the like. In some embodiments, an exemplary specifically programmed browser application of the present disclosure may be configured to receive and display graphics, text, multimedia, and the like, employing virtually any web based language, including, but not limited to Standard Generalized Markup Language (SMGL), such as HyperText Markup Language (HTML), a wireless application protocol (WAP), a Handheld Device Markup Language (HDML), such as Wireless Markup Language (WML), WMLScript, XML, JavaScript, and the like. In some embodiments, a member device within member devices 2002-2004 may be specifically programmed by either Java, .Net, QT, C, C++ and/or other suitable programming language. In some embodiments, one or more member devices within member devices 2002-2004 may be specifically programmed include or execute an application to perform a variety of possible tasks, such as, without limitation, messaging functionality, browsing, searching, playing, streaming or displaying various forms of content, including locally stored or uploaded messages, images and/or video, and/or games.

In some embodiments, the exemplary network 2005 may provide network access, data transport and/or other services to any computing device coupled to it. In some embodiments, the exemplary network 2005 may include and implement at least one specialized network architecture that may be based at least in part on one or more standards set by, for example, without limitation, Global System for Mobile communication (GSM) Association, the Internet Engineering Task Force (IETF), and the Worldwide Interoperability for Microwave Access (WiMAX) forum. In some embodiments, the exemplary network 2005 may implement one or more of a GSM architecture, a General Packet Radio Service (GPRS) architecture, a Universal Mobile Telecommunications System (UMTS) architecture, and an evolution of UMTS referred to as Long Term Evolution (LTE). In some embodiments, the exemplary network 2005 may include and implement, as an alternative or in conjunction with one or more of the above, a WiMAX architecture defined by the WiMAX forum. In some embodiments and, optionally, in combination of any embodiment described above or below, the exemplary network 2005 may also include, for instance, at least one of a local area network (LAN), a wide area network (WAN), the Internet, a virtual LAN (VLAN), an enterprise LAN, a layer 3 virtual private network (VPN), an enterprise IP network, or any combination thereof. In some embodiments and, optionally, in combination of any embodiment described above or below, at least one computer network communication over the exemplary network 2005 may be transmitted based at least in part on one of more communication modes such as but not limited to: NFC, RFID, Narrow Band Internet of Things (NBIOT), ZigBee, 3G, 4G, 5G, GSM, GPRS, WiFi, WiMax, CDMA, satellite and any combination thereof. In some embodiments, the exemplary network 2005 may also include mass storage, such as network attached storage (NAS), a storage area network (SAN), a content delivery network (CDN) or other forms of computer or machine readable media.

In some embodiments, the exemplary server 2006 or the exemplary server 2007 may be a web server (or a series of servers) running a network operating system, examples of which may include but are not limited to Microsoft Windows Server, Novell NetWare, or Linux. In some embodiments, the exemplary server 2006 or the exemplary server 2007 may be used for and/or provide cloud and/or network computing. Although not shown in FIG. 20, in some embodiments, the exemplary server 2006 or the exemplary server 2007 may have connections to external systems like email, SMS messaging, text messaging, ad content providers, etc. Any of the features of the exemplary server 2006 may be also implemented in the exemplary server 2007 and vice versa.

In some embodiments, one or more of the exemplary servers 2006 and 2007 may be specifically programmed to perform, in non-limiting example, as authentication servers, search servers, email servers, social networking services servers, SMS servers, IM servers, MMS servers, exchange servers, photo-sharing services servers, advertisement providing servers, financial/banking-related services servers, travel services servers, or any similarly suitable service-base servers for users of the member computing devices 2001-2004.

In some embodiments and, optionally, in combination of any embodiment described above or below, for example, one or more exemplary computing member devices 2002-2004, the exemplary server 2006, and/or the exemplary server 2007 may include a specifically programmed software module that may be configured to send, process, and receive information using a scripting language, a remote procedure call, an email, a tweet, Short Message Service (SMS), Multimedia Message Service (MMS), instant messaging (IM), internet relay chat (IRC), mIRC, Jabber, an application programming interface, Simple Object Access Protocol (SOAP) methods, Common Object Request Broker Architecture (CORBA), HTTP (Hypertext Transfer Protocol), REST (Representational State Transfer), or any combination thereof.

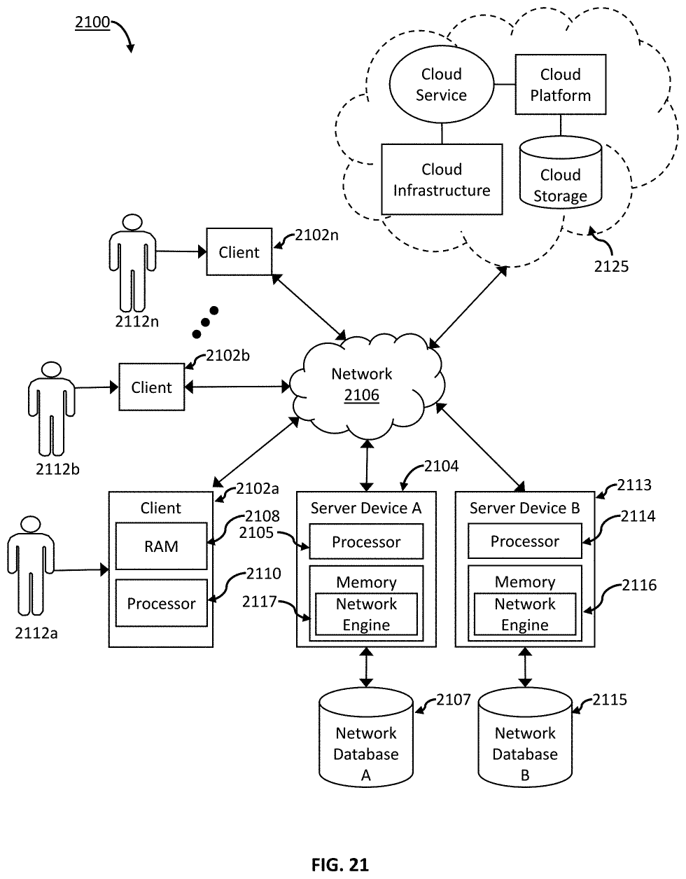

FIG. 21 depicts a block diagram of another exemplary computer-based system and platform 2100 in accordance with one or more embodiments of the present disclosure. However, not all of these components may be required to practice one or more embodiments, and variations in the arrangement and type of the components may be made without departing from the spirit or scope of various embodiments of the present disclosure. In some embodiments, the member computing devices 2102a, 2102b thru 2102n shown each at least includes a computer-readable medium, such as a random-access memory (RAM) 2108 coupled to a processor 2110 or FLASH memory. In some embodiments, the processor 2110 may execute computer-executable program instructions stored in memory 2108. In some embodiments, the processor 2110 may include a microprocessor, an ASIC, and/or a state machine. In some embodiments, the processor 2110 may include, or may be in communication with, media, for example computer-readable media, which stores instructions that, when executed by the processor 2110, may cause the processor 2110 to perform one or more steps described herein. In some embodiments, examples of computer-readable media may include, but are not limited to, an electronic, optical, magnetic, or other storage or transmission device capable of providing a processor, such as the processor 2110 of member computing device 2102a, with computer-readable instructions. In some embodiments, other examples of suitable media may include, but are not limited to, a floppy disk, CD-ROM, DVD, magnetic disk, memory chip, ROM, RAM, an ASIC, a configured processor, all optical media, all magnetic tape or other magnetic media, or any other medium from which a computer processor can read instructions. Also, various other forms of computer-readable media may transmit or carry instructions to a computer, including a router, private or public network, or other transmission device or channel, both wired and wireless. In some embodiments, the instructions may comprise code from any computer-programming language, including, for example, C, C++, Visual Basic, Java, Python, Perl, JavaScript, and etc.

In some embodiments, member computing devices 2102a through 2102n may also comprise a number of external or internal devices such as a mouse, a CD-ROM, DVD, a physical or virtual keyboard, a display, or other input or output devices. In some embodiments, examples of member computing devices 2102a through 2102n (e.g., clients) may be any type of processor-based platforms that are connected to a network 2106 such as, without limitation, personal computers, digital assistants, personal digital assistants, smart phones, pagers, digital tablets, laptop computers, Internet appliances, and other processor-based devices. In some embodiments, member computing devices 2102a through 2102n may be specifically programmed with one or more application programs in accordance with one or more principles/methodologies detailed herein. In some embodiments, member computing devices 2102a through 2102n may operate on any operating system capable of supporting a browser or browser-enabled application, such as Microsoft™, Windows™, and/or Linux. In some embodiments, member computing devices 2102a through 2102n shown may include, for example, personal computers executing a browser application program such as Microsoft Corporation's Internet Explorer™, Apple Computer, Inc.'s Safari™, Mozilla Firefox, and/or Opera. In some embodiments, through the member computing devices 2102a through 2102n, users, 2112a through 2102n, may communicate over the exemplary network 2106 with each other and/or with other systems and/or devices coupled to the network 2106. As shown in FIG. 21, exemplary server devices 2104 and 2113 may be also coupled to the network 2106. In some embodiments, one or more member computing devices 2102a through 2102n may be mobile clients.

In some embodiments, at least one database of exemplary databases 2107 and 2115 may be any type of database, including a database managed by a database management system (DBMS). In some embodiments, an exemplary DBMS-managed database may be specifically programmed as an engine that controls organization, storage, management, and/or retrieval of data in the respective database. In some embodiments, the exemplary DBMS-managed database may be specifically programmed to provide the ability to query, backup and replicate, enforce rules, provide security, compute, perform change and access logging, and/or automate optimization. In some embodiments, the exemplary DBMS-managed database may be chosen from Oracle database, IBM DB2, Adaptive Server Enterprise, FileMaker, Microsoft Access, Microsoft SQL Server, MySQL, PostgreSQL, and a NoSQL implementation. In some embodiments, the exemplary DBMS-managed database may be specifically programmed to define each respective schema of each database in the exemplary DBMS, according to a particular database model of the present disclosure which may include a hierarchical model, network model, relational model, object model, or some other suitable organization that may result in one or more applicable data structures that may include fields, records, files, and/or objects. In some embodiments, the exemplary DBMS-managed database may be specifically programmed to include metadata about the data that is stored.

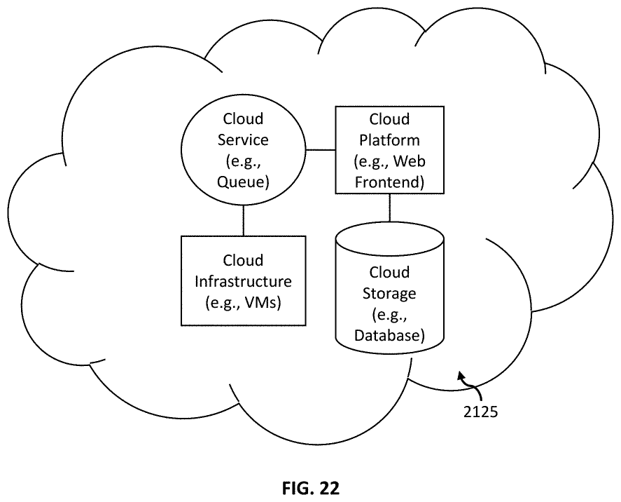

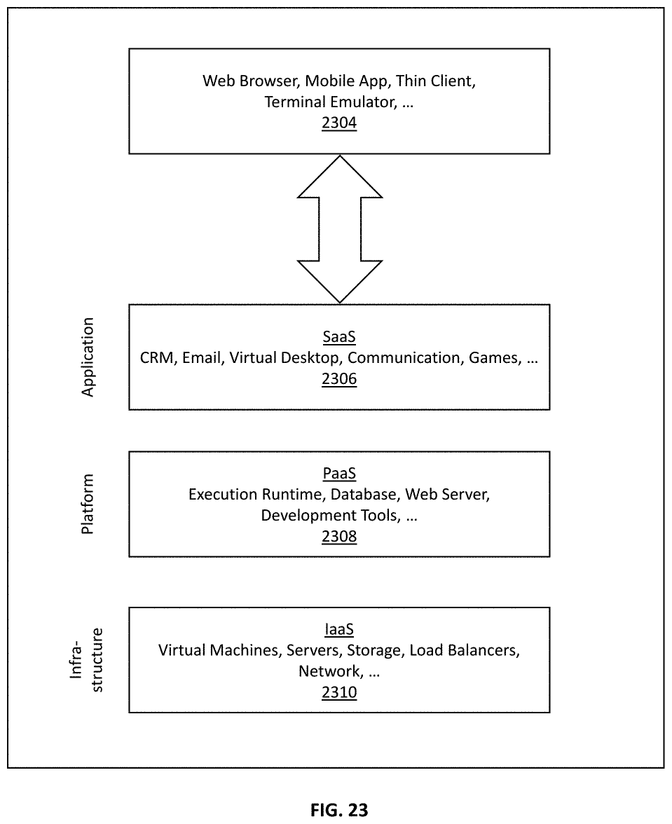

In some embodiments, the exemplary inventive computer-based systems/platforms, the exemplary inventive computer-based devices, and/or the exemplary inventive computer-based components of the present disclosure may be specifically configured to operate in a cloud computing/architecture 2125 such as, but not limiting to: infrastructure a service (IaaS) 2310, platform as a service (PaaS) 2308, and/or software as a service (SaaS) 2306 using a web browser, mobile app, thin client, terminal emulator or other endpoint 2304. FIGS. 22 and 23 illustrate schematics of exemplary implementations of the cloud computing/architecture(s) in which the exemplary inventive computer-based systems/platforms, the exemplary inventive computer-based devices, and/or the exemplary inventive computer-based components of the present disclosure may be specifically configured to operate.

### EXAMPLES

The following examples illustrate specific aspects of the instant description. The examples should not be construed as limiting, as the example merely provides specific understanding and practice of the embodiments and its various aspects.

### Example 1: Single-Cell Signal Using Amplification and Electrophoresis

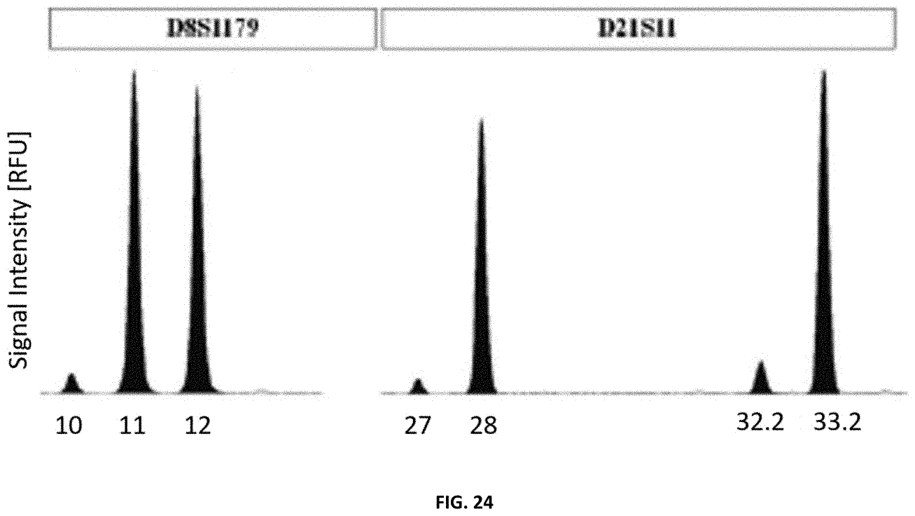

A 0.25 ng DNA sample was amplified (29 cycles) using Applied Biosystems™ GlobalFiler™ PCR Amplification Kit and an injection time of 10 sec using capillary electrophoresis on an Applied Biosystems® 3130 Genetic Analyzer (a capillary-based instrument). The laboratory technique of electrophoresis could be either capillary-based or gel-based. When electropherograms (EPGs) are generated using gels rather than capillaries the volume of liquid loaded into the gel can be taken to be analogous to the injection time (i.e., the more that is loaded or injected, the higher the peak height or area). See, FIG. 24 of example loci: D8S1179, D21S11. The X-axis represents the time it takes for the DNA fragment to reach a location in the capillary or gel, and therefore, represents the fragment size (in base pairs) of amplified product, which is a proxy for the allele at that particular locus (i.e., the further to the right the peak is, the larger the fragment). The Y-axis represents the signal intensity (i.e., in FIG. 24, Relative Fluorescent Units (RFU)), which is a proxy for the total number of DNA fragments. In brief, this method works with any instrument that records the signal intensity where the signal intensity is a proxy for the number of DNA fragments and records or report differences in DNA length.

### Example 2: Single-Cell Signal Using Amplification and NextGen Sequencing

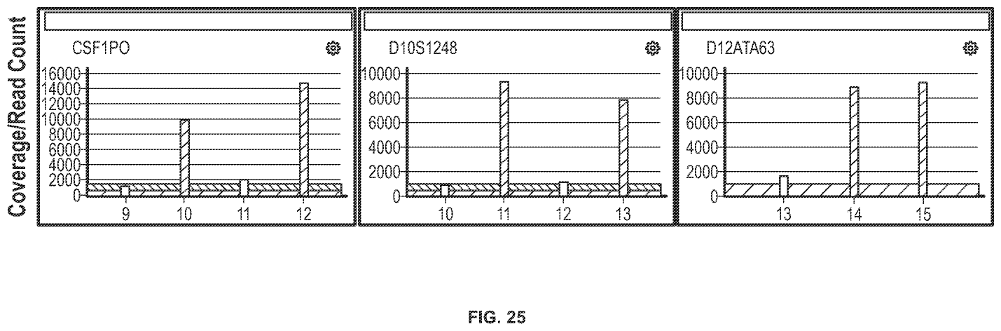

A 0.25 ng sample from a DNA library preparation using Applied Biosystems™ Precision ID GlobalFiler™ NGS STR Panel v2 was amplified (26 cycles) and an NGS concentration of 100 pM on an Ion Torrent next-generation sequencer (NGS) (ThermoFisher Scientific). See FIG. 25 of example loci: CSF1PO, D10S1248, D12ATA63. As with electropherograms (EPGs), this NGS readout of the signals or information from NGS systems is similar since signal intensity or absolute or relative coverage/read count is a proxy for the number of fragments, while the X-axis provides information on the length and sequence of the DNA fragment.

FIGS. 24 and 25 are analogous in that the Y-axis represents signal intensity (e.g., RFU or absolute counts) while the X-axis represents the STR (i.e., the base pair length of the fragment). Whether it be EGPs or NGS signal readouts, the total signal was composed of some combination of allele signal, artifact signal, and noise. Accordingly, the instrument or method in which signal intensity is obtained is not limiting as long as the signal represents the number of DNA fragments of a particular length or sequence.

### SPECIFIC EMBODIMENTS

Non-limiting specific embodiments are described below each of which is considered to be within the present disclosure.

In an example of aspects of embodiments of the present invention, the following description utilizes signal profiles includes EPGs to cluster single-cells for generating matching statistics. For single-cell DNA forensics, each peak of the EPG profile from a single cell can be thought of as a high-dimensional vector reporting the fluorescence measurement at each potential allele. A reasonable measure of similarity between two such vectors is the cosine distance and is zero if the vectors point in the same direction. As they point in increasingly discrepant directions, the distance increases up to a maximum distance of one. Using this distance, the similarity of the EPG signal from two cells is assessed not only by their fluorescence at true allele locations, but also at stutter locations and by the absence of fluorescence at other alleles.

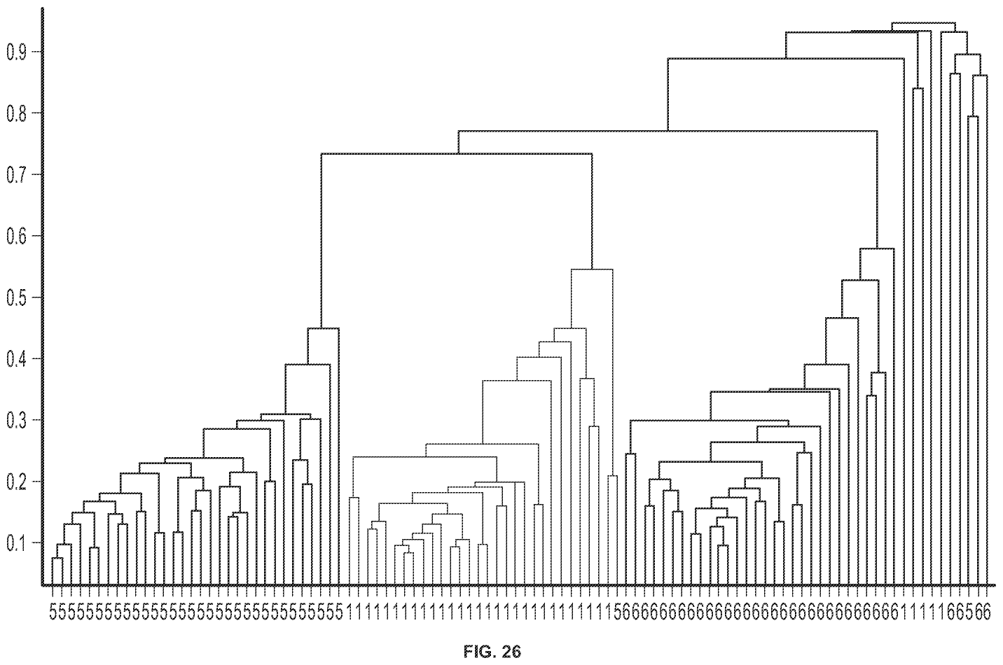

For the previously introduced data set, FIG. 26 plots the empirical density of Cosine Distance between EPGs created from cells of the same genotype (three lines of Self-Self distance distributions for Persons 01, 05 and 06) and from cells from distinct genotypes (three lines of Self-Non-Self, i.e., Cosine Distances between Persons 01 & 05; Persons 01 & 06 and Persons 05 & 06). While the distance between EPGs from the same genotype is typically smaller than distances between distinct genotypes, there is a long right tail indicating there are instances where the distance between two EPGs from the same genotype is as large, or larger, than from two distinct genotypes indicating that the two cases of Self-Self and Self-Non-Self cannot be unambiguously distinguished for these data.

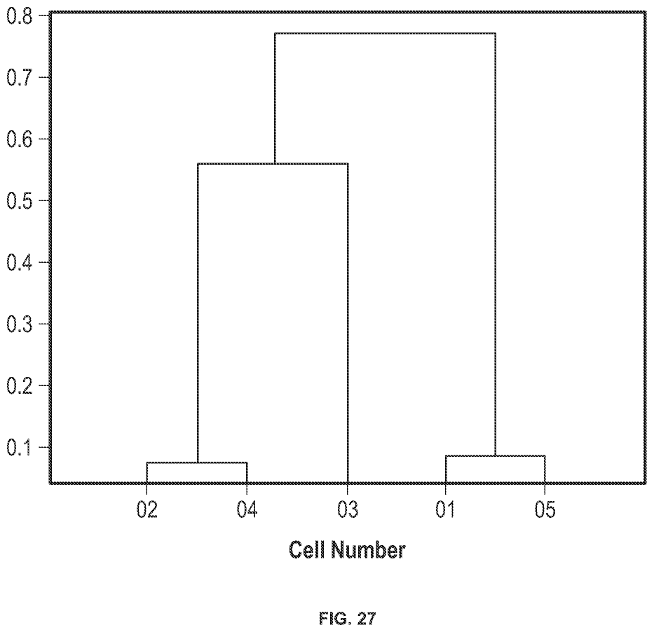

Agglomerative clustering is an unsupervised learning method that sequentially groups data points based on their similarity as determined by a measure of distances between them. Each data point begins in its own cluster and clusters are sequentially merged based on their similarity to form a complete hierarchy of relationships from most- to least-similar. This procedure results in a tree of nested groupings described by a dendrogram. FIG. 27 presents the outcome of performing clustering on these single cell data using cosine distance. The y-label is a measure of the dissimilarity between the two groups being joined at each stage in the dendrogram. While most of the EPGs from each of the individual genotypes form clusters, the initial branches of the dendrogram (reading from the top down), first separate ten EPGs taken from a variety of the contributors (5 from Person 01, 1 from Person 05 and 4 from Person 06). The expectation, which proves to be correct, is that these problematic EPGs constitute those that have few alleles identified above the analytical threshold; they are distant from EPGs of the any genotype because they contain little information. From an interpretation perspective, one must evaluate if these low-signal EPGs are to be explicitly modelled and included in any inference framework or filtered out.

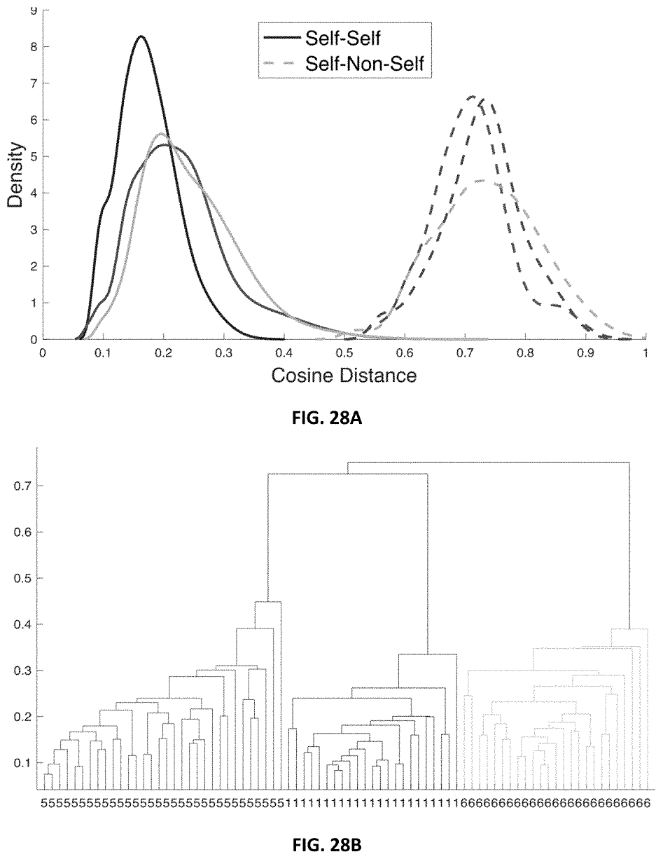

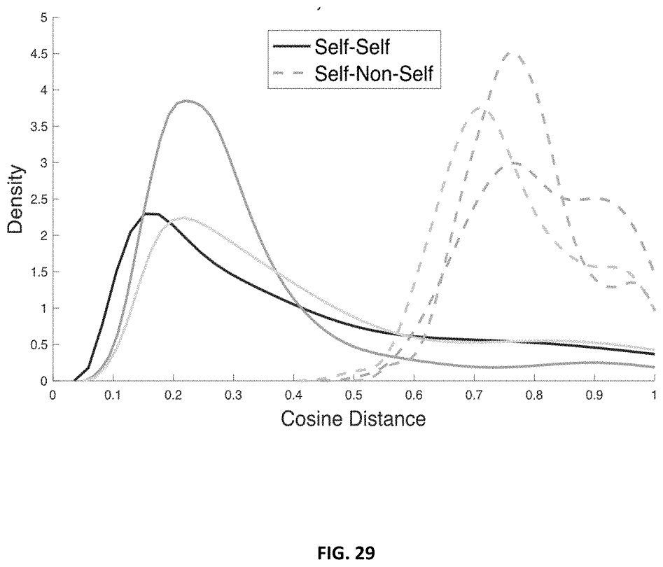

Low-quality signal from individual cells has been observed by other groups and is expected. One option would be to apply a naïve filtering rule set to remove low-quality EPGs from interpretation. As each EPG in this data is created from a single cell, one would anticipate that total signal RFU serves as a good proxy for the number alleles. To test if a high-pass total RFU filter sufficiently removes low-quality EPGs we apply a total RFU filter of 15,000 RFU (FIG. 28A) and replot the distribution of cosine distances. Despite the 15,000 RFU filter, most single-cell EPGs may still be available for interpretation (as suggested by FIG. 5 above) and EPGs that contain little genetic information are effectively removed prior to interpretation. When FIG. 28A is compared with the unfiltered data in FIG. 26, the long tails of the Self-Self distance distributions are absent, as are the second modes of the Self-Non-Self distance distributions and the primary branches of the dendrogram. FIG. 28B, now, correctly separate the genotypes.

In some embodiments, the dendrogram provides a hierarchy of nested grouping in terms of signal similarity but does not directly identify how many contributors there are. For that purpose, properties of DNA forensics signal may be leveraged where it is known that each individual should have no more than two alleles per locus, the population statistics of the alleles is known, and so forth. To that end, in some embodiments, starting from the root of the resulting dendrogram, NoC methodologies may be used to determine if there is more than one contributor to all signals found beneath that node. If there is more than one contributor, samples are divided according to sub-groupings at the next level of the dendrogram, which splits the samples into two groups with greatest dissimilarity, and this process is repeated recursively until the NoC to each group is one. The outcome of this procedure is both the NoC to the overall sample and the grouping of single cell signals per-contributor.

In some embodiments, the output of the pipeline described above is the determination of the NoC and groupings of single cell samples by contributor. For each group, one can then perform comparisons based on those samples with any existing methodology that describes the weight of evidence. The weight of evidence may focus on the likelihood ratio (LR). In some embodiments, either the average clustered signal per contributor or considering each cell, separately may be employed. For example, suppose that in a particular cluster there are clustered n EPGs, E₁, E₂, . . . , Eₙ, where each EPG Eᵢ is a vector of peak heights. From these EPGs, an average is produced by EPG Ê=Σᵢ₌₁ⁿEᵢ/n. Variants of traditional match statistics considered for single cells may be LR_{avg} and LRₛₑₚ, where

$$L {R}_{a v g} = \frac{P ( \hat{E} \vert {H}_{1} )}{P ( \hat{E} \vert {H}_{2} )} and \qquad ( Eq 13 )$$

$$L {R}_{s e p} = \frac{P ( {E}_{1} , {E}_{2} , … , {E}_{n} \vert {H}_{1} )}{P ( {E}_{1} , {E}_{2} , … , {E}_{n} \vert {H}_{2} )} \qquad ( Eq 14 )$$

Here, H₁ and H₂ might refer to the prosecution and defense hypotheses, specifically, which are generally assumed to be that the evidence (i.e. the EPGs) arises from the genotype of a specific POI for H₁ and that the evidence arises from the genotype of a random individual from the background population. In some embodiments, one of the most significant challenges in computing the LR is removed, because by design the average EPG Ê assumes to arises from a single contributor. The calculation of LRₛₑₚ is more challenging. To compute LRₛₑₚ, the conditional independence of each EPG may be utilized, given a particular genotype g that they all arise from. Specifically, let H₁(g) be the hypothesis that all EPGs arise from a contributor with genotype g, then

P(E₁,E₂, . . . ,Eₙ|H₁(g))=Πᵢ₌₁ⁿP(Eᵢ|H₁(g)) (Eq. 15)

The calculation of LRₛₑₚ may require more computational resources than the calculation of LR_{avg}.

In other embodiments, let L be the set of loci. Consider genotype g=(g₁, . . . , g_{L}) and ith electropherogram Eᵢ=(E_{i,1}, . . . , E_{i,L}), where gᵢ denotes the genotype at locus l∈L, E_{i,l} denotes the ith electropherogram at locus l∈L. Because of conditional independence of the electropherogram at each locus,

P(Eᵢ|H₁(g))=Π_{i∈L}P(E_{i,1}|H₁(g₁, . . . ,g_{L})) (Eq. 16)

Because of the conditional independence of the n electropherograms E₁, . . . , Eₙ, Pr(E|H₁(s)) may be calculated as

Pr(E|H₁(s))=Πᵢ₌₁^{m}Pr(Eᵢ|H₁(s))=Πᵢ₌₁^{m}Π_{l∈L}Pr(E_{i,l}|H₁(s₁, . . . s_{L}))=Π_{l∈L}Πᵢ₌₁^{m}Pr(E_{i,l}|H_{1,l}(sₗ)) (Eq. 17)

where Pr(E_{i,l}|H_{1,l}(sₗ)) is the probability of observing electropherogram E_{i,l} given a contributor with genotype sₗ at locus l, is calculated from the signal model Pr(E|H₂) is calculated using

Pr(E|H₂)=Πᵢ₌₁^{m}Σ_{g}Pr(Eᵢ|H₁(g))p_{G}(g) (Eq. 18)

where p_{G} is the probability mass function of genotypes G according to population frequencies.

Therefore:

Pr(E|H₂)=Πᵢ₌₁^{m}Σ_{g}_{₁}_{, . . . ,g}_{_{L}}Π_{l∈L}Pr(E_{i,l}|H₁(g₁, . . . ,g_{L}))p_{G}(g₁, . . . ,g_{L})=Π_{l∈L}Σ_{g}_{ₗ}Πᵢ₌₁^{m}Pr(E_{i,l}|H_{1,l}(gₗ))p_{G}_{ₗ}(gₗ) (Eq. 19)

where p_{G}_{ₗ} is the probability mass function of genotypes Gₗ at locus l according to population frequencies.

As various changes can be made in the above-described subject matter without departing from the scope and spirit of the present disclosure, it is intended that all subject matter contained in the above description, or defined in the appended claims, be interpreted as descriptive and illustrative of the present disclosure. Many modifications and variations of the present disclosure are possible in light of the above teachings. Accordingly, the present description is intended to embrace all such alternatives, modifications and variances which fall within the scope of the appended claims.

It is understood that at least one aspect/functionality of various embodiments described herein can be performed in real-time and/or dynamically. As used herein, the term “real-time” is directed to an event/action that can occur instantaneously or almost instantaneously in time when another event/action has occurred. For example, the “real-time processing,” “real-time computation,” and “real-time execution” all pertain to the performance of a computation during the actual time that the related physical process (e.g., a user interacting with an application on a mobile device) occurs, in order that results of the computation can be used in guiding the physical process.

As used herein, the term “dynamically” and term “automatically,” and their logical and/or linguistic relatives and/or derivatives, mean that certain events and/or actions can be triggered and/or occur without any human intervention. In some embodiments, events and/or actions in accordance with the present disclosure can be in real-time and/or based on a predetermined periodicity of at least one of: nanosecond, several nanoseconds, millisecond, several milliseconds, second, several seconds, minute, several minutes, hourly, several hours, daily, several days, weekly, monthly, etc.

In some embodiments, exemplary inventive, specially programmed computing systems and platforms with associated devices are configured to operate in the distributed network environment, communicating with one another over one or more suitable data communication networks (e.g., the Internet, satellite, etc.) and utilizing one or more suitable data communication protocols/modes such as, without limitation, IPX/SPX, X.25, AX.25, AppleTalk™, TCP/IP (e.g., HTTP), near-field wireless communication (NFC), RFID, Narrow Band Internet of Things (NBIOT), 3G, 4G, 5G, GSM, GPRS, WiFi, WiMax, CDMA, satellite, ZigBee, and other suitable communication modes.

The material disclosed herein may be implemented in software or firmware or a combination of them or as instructions stored on a machine-readable medium, which may be read and executed by one or more processors. A machine-readable medium may include any medium and/or mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Computer-related systems, computer systems, and systems, as used herein, include any combination of hardware and software. Examples of software may include software components, programs, applications, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, etc.).

In some embodiments, one or more of illustrative computer-based systems or platforms of the present disclosure may include or be incorporated, partially or entirely into at least one personal computer (PC), laptop computer, ultra-laptop computer, tablet, touch pad, portable computer, handheld computer, palmtop computer, personal digital assistant (PDA), cellular telephone, combination cellular telephone/PDA, television, smart device (e.g., smart phone, smart tablet or smart television), mobile internet device (MID), messaging device, data communication device, and so forth.

As used herein, term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

In some embodiments, as detailed herein, one or more of the computer-based systems of the present disclosure may obtain, manipulate, transfer, store, transform, generate, and/or output any digital object and/or data unit (e.g., from inside and/or outside of a particular application) that can be in any suitable form such as, without limitation, a file, a contact, a task, an email, a message, a map, an entire application (e.g., a calculator), data points, and other suitable data. In some embodiments, as detailed herein, one or more of the computer-based systems of the present disclosure may be implemented across one or more of various computer platforms such as, but not limited to: (1) Linux, (2) Microsoft Windows, (3) OS X (Mac OS), (4) Solaris, (5) UNIX (6) VMWare, (7) Android, (8) Java Platforms, (9) Open Web Platform, (10) Kubernetes or other suitable computer platforms. In some embodiments, illustrative computer-based systems or platforms of the present disclosure may be configured to utilize hardwired circuitry that may be used in place of or in combination with software instructions to implement features consistent with principles of the disclosure. Thus, implementations consistent with principles of the disclosure are not limited to any specific combination of hardware circuitry and software. For example, various embodiments may be embodied in many different ways as a software component such as, without limitation, a stand-alone software package, a combination of software packages, or it may be a software package incorporated as a “tool” in a larger software product.

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

In some embodiments, illustrative computer-based systems or platforms of the present disclosure may be configured to handle numerous concurrent users that may be, but is not limited to, at least 100 (e.g., but not limited to, 100-999), at least 1,000 (e.g., but not limited to, 1,000-9,999), at least 10,000 (e.g., but not limited to, 10,000-99,999), at least 100,000 (e.g., but not limited to, 100,000-999,999), at least 1,000,000 (e.g., but not limited to, 1,000,000-9,999,999), at least 10,000,000 (e.g., but not limited to, 10,000,000-99,999,999), at least 100,000,000 (e.g., but not limited to, 100,000,000-999,999,999), at least 1,000,000,000 (e.g., but not limited to, 1,000,000,000-999,999,999,999), and so on.

In some embodiments, illustrative computer-based systems or platforms of the present disclosure may be configured to output to distinct, specifically programmed graphical user interface implementations of the present disclosure (e.g., a desktop, a web app., etc.). In various implementations of the present disclosure, a final output may be displayed on a displaying screen which may be, without limitation, a screen of a computer, a screen of a mobile device, or the like. In various implementations, the display may be a holographic display. In various implementations, the display may be a transparent surface that may receive a visual projection. Such projections may convey various forms of information, images, or objects. For example, such projections may be a visual overlay for a mobile augmented reality (MAR) application.

In some embodiments, illustrative computer-based systems or platforms of the present disclosure may be configured to be utilized in various applications which may include, but not limited to, gaming, mobile-device games, video chats, video conferences, live video streaming, video streaming and/or augmented reality applications, mobile-device messenger applications, and others similarly suitable computer-device applications.

As used herein, terms “cloud,” “Internet cloud,” “cloud computing,” “cloud architecture,” and similar terms correspond to at least one of the following: (1) a large number of computers connected through a real-time communication network (e.g., Internet); (2) providing the ability to run a program or application on many connected computers (e.g., physical machines, virtual machines (VMs)) at the same time; (3) network-based services, which appear to be provided by real server hardware, and are in fact served up by virtual hardware (e.g., virtual servers), simulated by software running on one or more real machines (e.g., allowing to be moved around and scaled up (or down) on the fly without affecting the end user).

In some embodiments, the illustrative computer-based systems or platforms of the present disclosure may be configured to securely store and/or transmit data by utilizing one or more of encryption techniques (e.g., private/public key pair, Triple Data Encryption Standard (3DES), block cipher algorithms (e.g., IDEA, RC2, RC5, CAST and Skipjack), cryptographic hash algorithms (e.g., MD5, RIPEMD-160, RTRO, SHA-1, SHA-2, Tiger (TTH), WHIRLPOOL, RNGs).

As used herein, the term “user” shall have a meaning of at least one user. In some embodiments, the terms “user”, “subscriber” “consumer” or “customer” should be understood to refer to a user of an application or applications as described herein and/or a consumer of data supplied by a data provider. By way of example, and not limitation, the terms “user” or “subscriber” can refer to a person who receives data provided by the data or service provider over the Internet in a browser session, or can refer to an automated software application which receives the data and stores or processes the data.

The aforementioned examples are, of course, illustrative and not restrictive.

At least some aspects of the present disclosure will now be described with reference to the following numbered clauses:

Clause 1. A method comprising:

receiving, by at least one processor, a sample set of signal profiles;
wherein the signal profiles are associated with a plurality of cells of an admixture;
wherein each cell of the plurality of cells comprises a plurality of loci;
wherein each locus of the plurality of loci comprises a plurality of alleles;
wherein each allele comprises a magnitude of a measurements; for each cell of the plurality of cells:
determining, by the at least one processor, a set of vectors representing the magnitude of the measurement at each allele of each locus;
   wherein each vector of the set of vectors is associated with each locus of the plurality of loci;
   wherein the magnitude of the measurement at each allele is mapped to a predetermined index location in an associated vector of the set of vectors;
generating, by the at least one processor, a cell vector in a set of cell vectors by concatenating each vector associated with each locus of the plurality of loci;
   wherein the set of cell vectors represent the sample set of signal profiles;

utilizing, by the at least one processor, at least one cluster model to create at least one cluster of at least one subset of cell vectors of the set of cell vectors in order to group the signal profiles within the sample set of signal profiles;
wherein each cluster is associated with a contributor of at least one contributor;

determining, by the at least one processor, a first likelihood of each subset of cell vectors of the at least one subset of cell vectors given that a target contributor of the at least one contributor supplied genetic material based at least in part on a comparison of a target signal profile and each cluster;

determining, by the at least one processor, a second likelihood of each subset of cell vectors of the at least one subset of cell vectors given that the target contributor of the at least one contributor did not supply genetic material based at least in part on a comparison of the target signal profile and each cluster;

determining, by the at least one processor, a likelihood ratio based at least in part on a ratio of the first likelihood and the second likelihood; and

generating, by the at least one processor, at least one visualization on at least one computing device associated with at least one user, wherein the at least one visualization displays the likelihood ratio.

Clause 2. The method according to clause 1, further comprising:

determining, by at least one processor, a likely number of contributors based at least in part on the at least one cluster;

determining, by the at least one processor, that the likely number of contributors exceeds an amount of the at least one cluster; and

generating, by the at least one processor, at least one additional cluster from the at least one cluster.

Clause 3. The method according to clause 1, further comprising:

determining, by at least one processor, a likely number of contributors based at least in part on the at least one cluster;
wherein the at least one cluster is a plurality of clusters;

determining, by the at least one processor, that an amount of the plurality of clusters exceeds the likely number of contributors;

determining, by the at least one processor, a subset of the plurality of clusters that are associated with a single contributor; and

generating, by the at least one processor, a single cluster from the subset of the plurality of clusters.

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the disclosure.

1. A method comprising:
receiving, by at least one processor, a sample set of signal profiles;
wherein the signal profiles are associated with a plurality of cells of an admixture;
wherein each cell of the plurality of cells comprises a plurality of loci;
wherein each locus of the plurality of loci comprises a plurality of alleles;
wherein each allele comprises a magnitude of a measurements;
for each cell of the plurality of cells:
determining, by the at least one processor, a set of cell vectors representing the magnitude of the measurement at each allele of each locus;
wherein each vector of the set of cell vectors is associated with each locus of the plurality of loci;
wherein the magnitude of the measurement at each allele is mapped to a predetermined index location in an associated vector of the set of cell vectors;
generating, by the at least one processor, a cell vector in a set of cell vectors by concatenating each vector associated with each locus of the plurality of loci;
wherein the set of cell vectors represent the sample set of signal profiles;
utilizing, by the at least one processor, at least one cluster model to create at least one cluster of at least one subset of cell vectors of the set of cell vectors in order to group the signal profiles within the sample set of signal profiles;
wherein each cluster is associated with a contributor of at least one contributor;
determining, by the at least one processor, a first likelihood of each subset of cell vectors of the at least one subset of cell vectors given that a target contributor of the at least one contributor supplied genetic material based at least in part on a comparison of a target signal profile and each cluster;
determining, by the at least one processor, a second likelihood of each subset of cell vectors of the at least one subset of cell vectors given that the target contributor of the at least one contributor did not supply genetic material based at least in part on a comparison of the target signal profile and each cluster;
determining, by the at least one processor, a likelihood ratio based at least in part on a ratio of the first likelihood and the second likelihood; and
generating, by the at least one processor, at least one visualization on at least one computing device associated with at least one user, wherein the at least one visualization displays the likelihood ratio.

2. The method of claim 1, further comprising:
determining, by at least one processor, a likely number of contributors based at least in part on the at least one cluster;
determining, by the at least one processor, that the likely number of contributors exceeds an amount of the at least one cluster; and
generating, by the at least one processor, at least one additional cluster from the at least one cluster.

3. The method of claim 1, further comprising:
determining, by at least one processor, a likely number of contributors based at least in part on the at least one cluster;
wherein the at least one cluster is a plurality of clusters;
determining, by the at least one processor, that an amount of the plurality of clusters exceeds the likely number of contributors;
determining, by the at least one processor, a subset of the plurality of clusters that are associated with a single contributor; and
generating, by the at least one processor, a single cluster from the subset of the plurality of clusters.

4. The method of claim 1, further comprising normalizing, by the at least one processor, the set of cell vectors based at least in part on a log-normal distribution.

5. The method of claim 1, wherein the at least one cluster model comprises at least one mixture model.

6. The method of claim 5, further comprising utilizing, by the at least one processor, the at least one mixture model to model the at least one cluster according to at least one probability distribution.

7. The method of claim 6, wherein the at least one probability distribution comprises at least one Gaussian distribution.

8. The method of claim 1, further comprising estimating, by the at least one processor, parameters of the at least one cluster model based at least in part on an expectation-maximization algorithm.

9. The method of claim 1, wherein each vector of the set of cell vectors encodes:
a true allele signal associated with a signal profile in the sample set of signal profiles,
a noise associated with the signal profile in the sample set of signal profiles, and
a reverse stutter associated with the signal profile in the sample set of signal profiles.

10. The method of claim 1, further comprising:
utilizing, by the at least one processor, a Uniform Manifold Approximation and Projection model to generate a high dimensional graph representation of the at least one cluster of the at least one subset of cell vectors; and
generating, by the at least one processor, at least one visualization comprising the high dimensional graph representation.

11. A system comprising:
at least one processor configured to perform steps to:
receive a sample set of signal profiles;
wherein the signal profiles are associated with a plurality of cells of an admixture;
wherein each cell of the plurality of cells comprises a plurality of loci;
wherein each locus of the plurality of loci comprises a plurality of alleles;
wherein each allele comprises a magnitude of a measurements;
for each cell of the plurality of cells:
determine a set of cell vectors representing the magnitude of the measurement at each allele of each locus;
wherein each vector of the set of cell vectors is associated with each locus of the plurality of loci;
wherein the magnitude of the measurement at each allele is mapped to a predetermined index location in an associated vector of the set of cell vectors;
generate a cell vector in a set of cell vectors by concatenating each vector associated with each locus of the plurality of loci;
wherein the set of cell vectors represent the sample set of signal profiles;
utilize at least one cluster model to create at least one cluster of at least one subset of cell vectors of the set of cell vectors in order to group the signal profiles within the sample set of signal profiles;
wherein each cluster is associated with a contributor of at least one contributor;
determine a first likelihood of each subset of cell vectors of the at least one subset of cell vectors given that a target contributor of the at least one contributor supplied genetic material based at least in part on a comparison of a target signal profile and each cluster;
determine a second likelihood of each subset of cell vectors of the at least one subset of cell vectors given that the target contributor of the at least one contributor did not supply genetic material based at least in part on a comparison of the target signal profile and each cluster;
determine a likelihood ratio based at least in part on a ratio of the first likelihood and the second likelihood; and
generate at least one visualization on at least one computing device associated with at least one user, wherein the at least one visualization displays the likelihood ratio.

12. The system of claim 11, wherein the at least one processor is further configured to perform steps to:
determining, by at least one processor, a likely number of contributors based at least in part on the at least one cluster;
determine that the likely number of contributors exceeds an amount of the at least one cluster; and
generate at least one additional cluster from the at least one cluster.

13. The system of claim 11, wherein the at least one processor is further configured to perform steps to:
determining, by at least one processor, a likely number of contributors based at least in part on the at least one cluster;
wherein the at least one cluster is a plurality of clusters;
determine that an amount of the plurality of clusters exceeds the likely number of contributors;
determine a subset of the plurality of clusters that are associated with a single contributor; and
generate a single cluster from the subset of the plurality of clusters.

14. The system of claim 11, wherein the at least one processor is further configured to perform steps to normalize the set of cell vectors based at least in part on a log-normal distribution.

15. The system of claim 11, wherein the at least one cluster model comprises at least one mixture model.

16. The system of claim 15, wherein the at least one processor is further configured to perform steps to utilize the at least one mixture model to model the at least one cluster according to at least one probability distribution.

17. The system of claim 16, wherein the at least one probability distribution comprises at least one Gaussian distribution.

18. The system of claim 11, wherein the at least one processor is further configured to perform steps to estimate parameters of the at least one cluster model based at least in part on an expectation-maximization algorithm.

19. The system of claim 11, wherein each vector of the set of cell vectors encodes:
a true allele signal associated with a signal profile in the sample set of signal profiles,
a noise associated with the signal profile in the sample set of signal profiles, and
a reverse stutter associated with the signal profile in the sample set of signal profiles.

20. The system of claim 11, wherein the at least one processor is further configured to perform steps to:
utilize a Uniform Manifold Approximation and Projection model to generate a high dimensional graph representation of the at least one cluster of the at least one subset of cell vectors; and
generate at least one visualization comprising the high dimensional graph representation.
